(19) United States
(12) Patent Application Publication
(10) Pub. No.: US 2024/0384191 A1
(43) Pub. Date: Nov. 21, 2024

(54) SYNGAS AND LIQUID FUEL PRODUCTION FROM OLIGOMERIZATION BYPRODUCTS

(71) Applicant: UOP LLC, Des Plaines, IL (US)

(72) Inventors: Joel S. Paustian, Vernon Hills, IL (US); Ian G. Horn, Streamwood, IL (US)

(21) Appl. No.: 18/653,881

(22) Filed: May 2, 2024

Related U.S. Application Data
(60) Provisional application No. 63/466,626, filed on May 15, 2023.

(51) Int. Cl. C10L 1/08; B01D 3/14; C01B 3/36; C07C 1/04; C07C 1/12; C10J 1/00

(52) U.S. Cl. CPC C10L 1/08; B01D 3/143; C01B 3/36; C07C 1/04; C07C 1/12; C10J 1/00; C01B 2203/0205; C01B 2203/025; C01B 2203/062; C10J 2300/0913; C10L 2200/0446

(57) ABSTRACT

A process for the production of a liquid fuel from carbon oxides and hydrogen is disclosed. The process comprises reacting a mixture of carbon oxides and hydrogen to produce methanol. The methanol is contacted with an MTO catalyst to produce an olefin stream. The olefin stream is oligomerized with an oligomerization catalyst to produce an oligomerized olefin stream which is then hydrogenated to produce the liquid fuel streams jet fuel, diesel, and naphtha. At each stage, waste streams are produced. A syngas stream is produced comprising carbon oxides and hydrogen by (1) steam reforming, autothermal reforming, or dry reforming; or (2) partial oxidization.

### FIELD

The field is related to a process for producing liquid fuel from carbon oxides and hydrogen. The field may particularly relate to a process for converting syngas to liquid fuel such as naphtha, jet fuel or diesel.

### BACKGROUND

The global need for low carbon intensity liquid fuels compatible with existing infrastructure is driving increased research and investment in fuel refining facilities utilizing CO2 as a primary feedstock. Within such facilities, CO2 is reacted with hydrogen (perhaps generated from water electrolysis) to produce methanol, which may be refined to produce fuels meeting the physical and chemical property requirements of gasoline, jet fuel, or diesel. The fuels derive their energy from renewable electricity (termed “eFuels”) and are envisioned to dramatically reduce the lifecycle carbon footprint of vehicles utilizing conventional combustion-powered engines. The eFuel production process may exist in various forms, one of which includes a water electrolysis unit, a methanol synthesis unit, and a methanol to jet (MTJ) unit. There may also be CO2 capture.

The process which is most desirable to the marketplace will produce high fuel yield with low carbon intensity. Efficient utilization of both light and heavy byproducts is essential in achieving this Process configurations which recycle waste streams in efficient manners will generate high yields of liquid fuels from carbon dioxide and hydrogen while minimizing incremental CO2 generation.

### BRIEF SUMMARY

We have formulated a process for producing liquid fuels such as naphtha, jet fuel or diesel with an improvement in the overall selectivity of the CO2 to jet complex (and a reduction in the carbon intensity). The improvement comprises recycling lighter and heavier hydrocarbons obtained in the process through reforming or partial oxidation processes.

### BRIEF DESCRIPTION OF THE DRAWINGS

FIGS. 1-3 are different embodiments of a carbon oxides to jet fuel complex showing principal processing blocks, interconnecting streams, feeds, and product and including the recycling of waste streams of the present disclosure.

FIG. 4 is a schematic drawing of a methanol synthesis process and apparatus of the present disclosure.

FIG. 5 is a schematic drawing of an olefinic feed preparation process and apparatus of the present disclosure.

FIG. 6 is an schematic drawing of an embodiment of oligomerization feed preparation section of the process and apparatus of the present disclosure.

FIG. 7 is a schematic drawing of an oligomerization process and apparatus of the present disclosure.

FIG. 8 is a schematic drawing of a hydrogenation process and apparatus of the present disclosure.

FIG. 9 is a schematic drawing of a partial oxidation section of a process and apparatus of the present disclosure.

FIG. 10 is a graph showing ethylene and propylene conversion vs hours on stream in accordance with an exemplary embodiment of the present disclosure.

FIG. 11 is a graph showing selectivity of various hydrocarbons vs hours on stream in accordance with another exemplary embodiment of the present disclosure.

FIG. 12 is a graph showing selectivity of various hydrocarbons vs hours on stream in accordance with yet another exemplary embodiment of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take main product from the bottom.

As used herein, the term “diesel” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method or a T90 between 280° C. (536° F.) and about 340° C. (644° F.) using ASTM D-86. The term “green diesel” means diesel comprising hydrocarbons not sourced from fossil fuels.

As used herein, the term “green hydrogen” is hydrogen produced from a non-fossil-fuel source, typically by a water hydrolysis unit.

As used herein, the term “T5”, “T10”, “T90” or “T95” means the temperature at which 5 percent by mass or volume, 10 percent by mass or volume, 90 percent by mass or volume or 95 percent by mass or volume, as the case may be, respectively, of the sample boils using ASTM D-86 or TBP.

As used herein, the term “end point” (EP) means the temperature at which the sample has all boiled off using ASTM D-7169, ASTM D-86 or TBP, as the case may be.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

As used herein, the term “predominant” or “predominate” means greater than 50%, suitably greater than 75% and preferably greater than 90%.

As used herein, the term “a component-rich stream” or “rich stream” means that the rich stream coming out of a vessel has a greater concentration of the component than the feed to the vessel and preferably than all other streams withdrawn from the vessel.

As used herein, the term “a component-lean stream” or “lean stream” means that the lean stream coming out of a vessel has a smaller concentration of the component than the feed to the vessel and preferably than all other streams withdrawn from the vessel.

As used herein, the term “rich” means greater than 50%, suitably greater than 75% and preferably greater than 90%.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

The process and apparatus disclosed involves the production of a liquid fuel from carbon oxide and hydrogen. The process comprises reacting a mixture of carbon oxide and hydrogen to produce methanol, water, and a gaseous waste stream containing hydrogen, carbon monoxide, and dimethyl ether (DME). The methanol is contacted with an MTO catalyst and concentrated to produce one or more olefin streams and a gaseous waste stream containing hydrogen, carbon monoxide, and methane. The one or more olefin streams are oligomerized with one or more oligomerization catalysts to produce an oligomerized olefin stream comprising light to heavy olefins and a gaseous waste stream containing hydrogen, ethane, propane, and light olefins. The oligomerized olefin stream and, in some embodiments, a portion of the light olefins are reacted with hydrogen in the presence of a hydrogenation catalyst to produce jet fuel, diesel fuel, naphtha, and a gaseous waste stream containing hydrogen, propane, butane, and other light hydrocarbons.

The combination of the MTO, oligomerization and hydrogenation processes as described above results in a very high (>75%) selectivity of jet fuel. However, the value of the byproducts, diesel, naphtha, and waste gases, will depend on whether they can be used effectively as fuels. In situations where these streams cannot be used as fuels, it would be more economical to recycle them so that they can be converted into jet fuel. There are a number of pathways for this recycle to happen, each of which ultimately converts the non-desired hydrocarbons to a mixture of carbon oxides and hydrogen (syngas), which can be fed to the methanol synthesis unit to convert into methanol. The advantage of these pathways is that they create syngas without requiring hydrogen from the water electrolysis which is the most energy intensive part of the CO2 to jet complex.

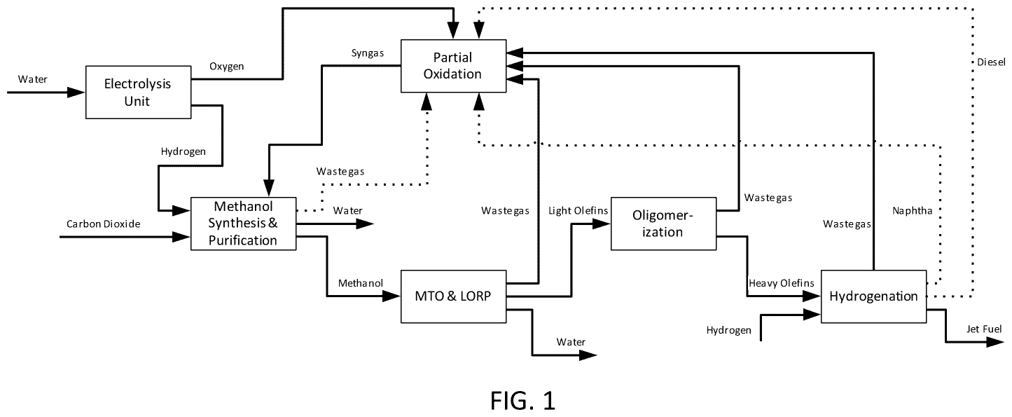

In one embodiment, oxygen from a water electrolysis process may be introduced in a partial-oxidation process to convert the non-desired by-products into syngas, which may then be used as a ready feed for the methanol synthesis unit. While both thermal and catalytic partial oxidation processes would be appropriate in this embodiment, the catalytic process would be preferred due to the fact that the reactants would be free of contaminants for that catalyst. See FIG. 1.

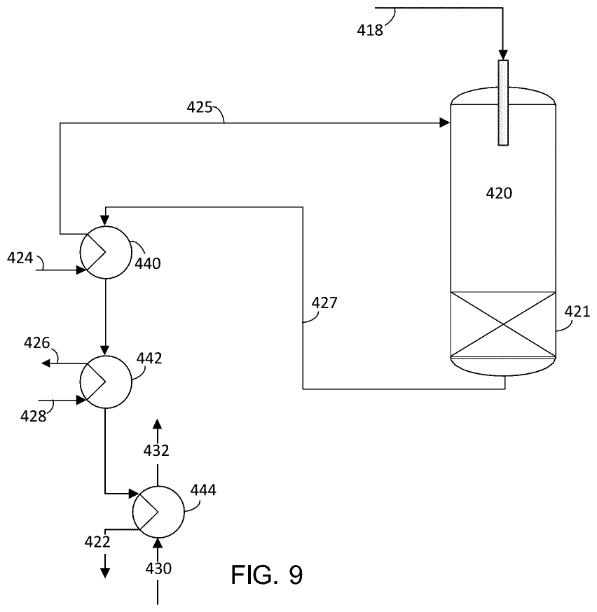

The partial oxidation process is exothermic and would provide its own heat to warm up the reactants. In a specific embodiment of the partial oxidation process, the process would provide additional heat to the hydrogenation process to be used in the reboiler of the distillation columns. See, e.g., FIG. 9.

In another embodiment, methane and hydrogen produced by the process in certain gaseous waste streams can be mixed with CO2 and reacted without the use of steam in a dry reforming process. This requires a non-precious metal-based catalyst developed by Linde and BASF. The resulting syngas can be used to make methanol. It may be possible that other light hydrocarbons in small amounts could also be processed this way.

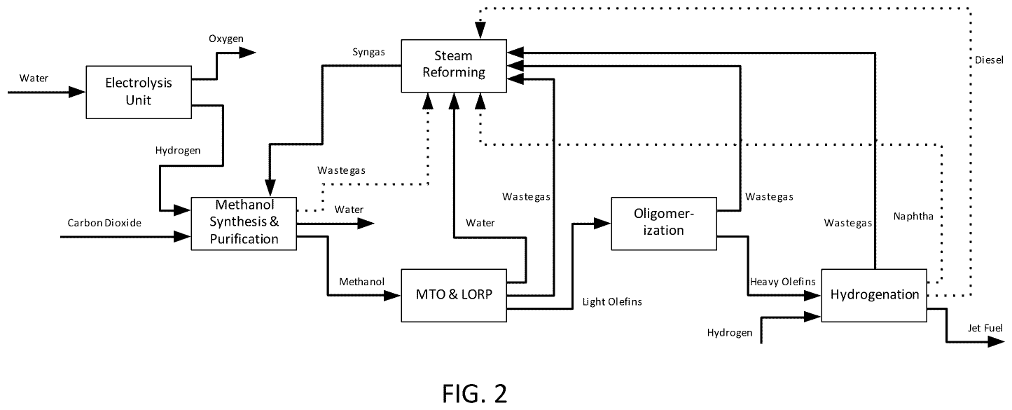

In another embodiment, the waste streams are reacted with steam in a steam reforming process at high temperatures to create a syngas mixture that can be fed directly to methanol synthesis. This process may include a gas heated reactor to improve overall yields. This is an endothermic process so requires a substantial amount of external heat. See FIG. 2.

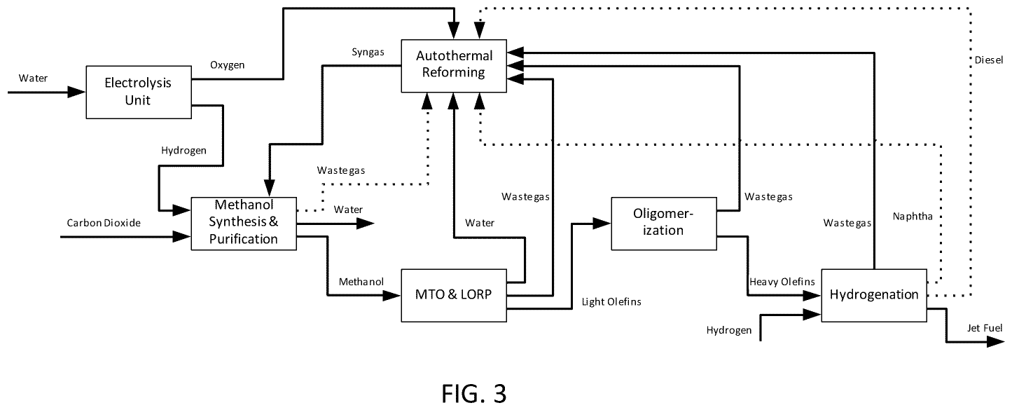

In yet another embodiment, autothermal reforming combines both partial oxidation and steam reforming. Oxygen from the water electrolysis unit, steam, and the waste streams are reacted over a catalyst to produce an appropriate syngas mixture for conversion to methanol. Heat for the process comes from the partial oxidation with oxygen. See FIG. 3.

The following methods and apparatus for methanol synthesis olefinic feed preparation, oligomerization and hydrogenation are given by way of example and not limitation. Other methods and apparatus for carrying out these processes may be used.

### Methanol Synthesis

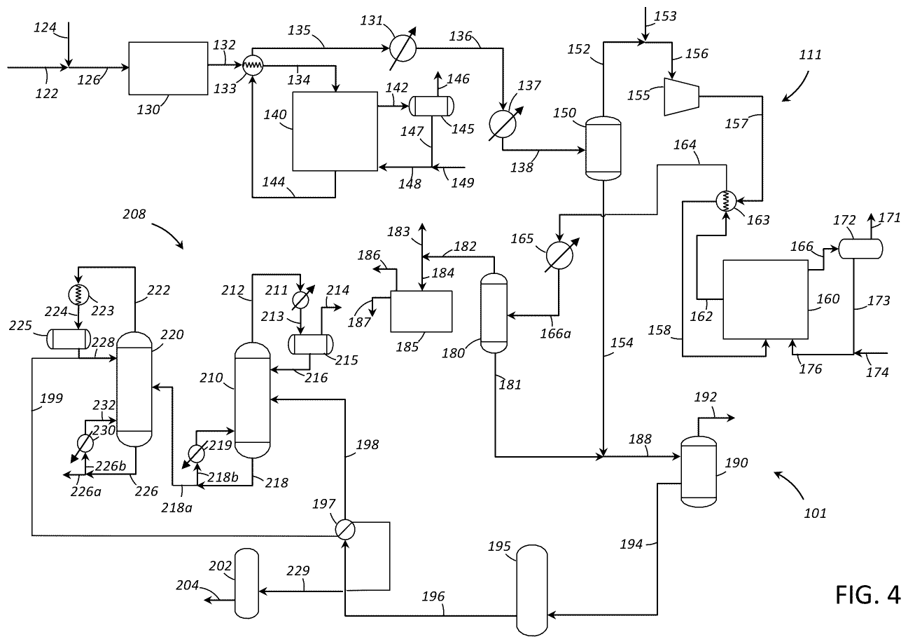

Turning to FIG. 4 of an integrated process and apparatus 101 for producing methanol comprises a methanol synthesis section 111 and a methanol purification section 208. The methanol synthesis section 111 may comprise reacting a mixture of carbon oxides and hydrogen to produce methanol, water, and a gaseous waste stream including hydrogen, carbon oxides, and dimethyl ether (DME).

In an exemplary embodiment as shown in FIG. 4, a syngas stream in line 122 and a hydrogen gas stream in line 124 are passed to the methanol synthesis section 111. Syngas is defined as a gas comprising primarily carbon oxides including carbon monoxide (CO), and carbon dioxide (CO2), and hydrogen (H2). Optionally, syngas may also include methane (CH4), and small amounts of ethane and propane. Conventional processes for converting carbon components to syngas include steam reforming, partial oxidation, autothermal reforming, and combinations of these processes. In accordance with an embodiment of the present disclosure, the syngas stream in line 122 may be taken from any suitable sources. In accordance with another embodiment of the present disclosure, the hydrogen gas stream in line 124 may be taken from any suitable sources. In an exemplary embodiment, the hydrogen gas stream in line 124 is produced by a water electrolysis unit.

In accordance with an exemplary embodiment, of the present disclosure, the methanol synthesis section 111 comprises a first methanol converter 140 and a second methanol converter 160. The syngas stream in line 122 and the hydrogen gas stream in line 124 are passed to the first methanol converter 140 of the methanol synthesis section 111. In an embodiment, the syngas stream in line 122 and the hydrogen gas stream in line 124 may be combined to provide a combined feed stream 126 which is passed to the first methanol converter 140. However, the syngas stream in line 122 and the hydrogen gas stream in line 124 may be passed separately to the first methanol converter 140. The combined feed stream 126 may be passed to a syngas pressure booster compressor 130 to compress the syngas to a particular pressure to provide a compressed syngas stream in line 132 before passing to the first methanol converter 140. In an exemplary embodiment, the syngas may be compressed to a pressure from about 6890 kPa (1000 psia) to about 8970 kPa (1300 psia) in the syngas pressure booster compressor 130. The syngas stream may be heated before passing it to the first methanol converter 140. The compressed syngas stream in line 132 may be heat exchanged in a heat exchanger 133 with a first reactor effluent stream in line 144 to provide a heated syngas stream in line 134. The heated syngas stream in line 134 is passed to the first methanol converter 140.

In the first methanol converter 140 of the methanol synthesis section 111, the syngas is converted to a methanol composition. The methanol synthesis process is accomplished in the presence of a methanol synthesis catalyst. In an exemplary embodiment, the syngas stream in line 122 to the methanol synthesis section 111 has a molar ratio of carbon dioxide to carbon monoxide of between 1:2 and 1:4 and a molar ratio of hydrogen to carbon oxides (CO+CO2) in the range of from about 3:2 to about 3:1.

A suitable methanol synthesis catalyst may be a copper on a zinc oxide and alumina support. Synthesis conditions of the first methanol converter 140 of the methanol synthesis section 111 may include a temperature of about 200 to about 300° C. and a pressure of about 3.5 to about 10 MPa. Reaction equilibrium typically requires methanol separation and recycle of unreacted reagents to the synthesis reaction to obtain sufficient conversion.

In accordance with an exemplary embodiment, the first methanol converter 140 may operate at a temperature of about 204° C. (400° F.) to about 290° C. (550° F.). In accordance with another exemplary embodiment, the first methanol converter 140 may operate at a pressure from about 6890 kPa (1000 psia) to about 8970 kPa (1300 psia).

The methanol synthesis reaction is highly exothermic. A boiler feed water (BFW) in line 148 is passed to the first methanol converter 140 to generate a steam stream in line 142, which is withdrawn from the first methanol converter 140. The generation of steam absorbs the exotherm in the methanol synthesis reaction. The steam stream in line 142 is passed to a steam separator 145 to separate steam in line 146 from a water stream in line 147. The water stream in line 147 is supplemented with a recycled BFW in line 149 to provide the BFW in line 148 for the first methanol converter 140. The boiler feed water in line 148 and steam in line 142 are separated from the heated syngas stream in line 134 by tubes or jackets in the first methanol converter 140 such that heat from the syngas reaction can indirectly transfer to the water.

In the first methanol converter 140, the syngas is converted to a methanol composition in a first reactor effluent comprising methanol in line 144. The methanol stream in the first reactor effluent stream in line 144 may include methanol, dimethyl ether, ethanol or combinations thereof. The first reactor effluent in line 144 is heat exchanged in the heat exchanger 133 with the compressed syngas stream in line 132. A first cooled first reactor effluent in line 135 may be further cooled in a first cooler 131 to provide a second cooled first reactor effluent stream in line 136. The second cooled first reactor effluent stream in line 136 may be further cooled in a second cooler 137 to provide a third cooled first reactor effluent stream in line 138. The third cooled first reactor effluent stream in line 138 is separated in a first gas-liquid separator 150 to provide a first vapor stream in line 152 and a first liquid stream in line 154. The first vapor stream in line 152 and the first liquid stream in line 154 may be further processed to recover methanol. The first vapor stream in line 152 comprises carbon dioxide that has not yet converted to methanol. The first vapor stream in line 152 may be compressed in a first compressor 155. In an embodiment, the first vapor stream in line 152 may be combined with a make-up hydrogen stream in line 153 to provide a combined first vapor stream in line 156. The combined first vapor stream in line 156 is compressed in the first compressor 155 to provide a compressed first vapor stream in line 157 at a pressure from about 6890 kPa (1000 psia) to about 8970 kPa (1300 psia). In an embodiment, the make-up hydrogen stream in line 153 may be taken from any suitable sources. In accordance with the present disclosure, the make-up hydrogen stream in line 153 may be taken from one or more units of the process 101.

The compressed first vapor stream in line 157 is heat exchanged with a second reactor effluent stream in the heat exchanger 163 to provide a heated first vapor stream in line 158 which is passed to the second methanol converter 160. In the second methanol converter 160 of the methanol synthesis section 111, the unconverted carbon dioxide in the syngas is converted to a methanol composition. The methanol synthesis process is accomplished in the presence of a methanol synthesis catalyst. A suitable methanol synthesis catalyst may be a copper on a zinc oxide and alumina support. Synthesis conditions of the second methanol converter 160 of the methanol synthesis section 111 may include a temperature of about 200 to about 300° C. and a pressure of about 3.5 to about 10 MPa. Reaction equilibrium typically requires methanol separation and recycle of unreacted reagents to the synthesis reaction.

A boiler feed water (BFW) in line 176 is passed to the second methanol converter 160 to generate a steam stream in line 166 withdrawn from the second methanol converter 160 to manage the exotherm. The steam stream in line 166 is passed to a steam separator 172 to separate steam in line 171 from a water stream in line 173. The water stream in line 173 is supplemented with a recycled BFW in line 174 to provide the BFW in line 176 for the second methanol converter 160. The boiler feed water in line 176 and the steam stream in line 166 are separated from the heated first vapor stream in line 158 by tubes or jackets in the second methanol converter 160 such that heat from the second methanol converter reaction can indirectly transfer to the water.

In the second methanol converter 160, the first reactor effluent stream is converted to a methanol composition to provide a second reactor effluent stream comprising methanol in line 162. The methanol stream in the second reactor effluent stream in line 162 may include methanol, dimethyl ether, ethanol or combinations thereof. The second reactor effluent stream in line 162 may be withdrawn from a side of the second methanol converter 160. The second reactor effluent stream in line 162 is cooled in the heat exchanger 163 with the compressed first vapor in line 157. A first cooled second reactor effluent stream in line 164 may be further cooled in a cooler 165 to provide a second cooled second reactor effluent stream in line 166a. The second cooled second reactor effluent stream in line 166a is separated in a second gas-liquid separator 180 to provide a second vapor stream in line 182 and a second liquid stream in line 184. The second vapor stream in line 182 and the second liquid stream in line 184 may be further processed to recover methanol. In an exemplary embodiment, the second vapor stream in line 182 may comprise hydrogen and carbon oxides that were not converted in methanol converters 140 and 160. It may also comprise one or more by-product components, including methane, methanol, ethers, particularly dimethyl ether, nitrogen, and oxygen.

In accordance an exemplary embodiment, the second methanol converter 160 is operated at a temperature of about 204° C. (400° F.) to about 290° C. (550° F.). In accordance with another exemplary embodiment, the second methanol converter 160 is operated at a pressure from about 6890 kPa (1000 psia) to about 8970 kPa (1300 psia).

In accordance with the present disclosure, the second vapor stream in line 182 is passed to a PSA unit 185 to separate hydrogen from the second vapor stream in line 182. In an exemplary embodiment, the second vapor stream in line 182 may be separated into a recycle stream in line 183 and a PSA feed stream in line 184. In another exemplary embodiment, the recycle stream in line 183 may be passed to the first compressor 155 as the make-up hydrogen stream in line 153. In an embodiment, the make-up hydrogen stream in line 153 to the first compressor 155 comprises the recycle stream in line 183. In an exemplary embodiment, 95% of the second vapor stream in line 182 is recycled to the makeup hydrogen stream in line 153. In other exemplary embodiments, between about 80% and about 98% of the second vapor stream in line 182 is recycled to the makeup hydrogen stream in line 153.

The PSA feed stream in line 184 is processed in the PSA unit 185. Typically, PSA unit includes a series of multiple adsorber beds containing one or a combination of multiple adsorbents suitable for adsorbing the particular components to be adsorbed therein. These adsorbents include, but are not limited to activated alumina, silica gel, activated carbon, zeolite molecular sieve type materials, or any combination thereof. The adsorbents are organized in any sequence as required by the adsorption process to adsorb impurities or components. In the PSA unit 185, PSA feed stream in line 184 flows over the adsorbents and the more readily adsorbable components are adsorbed during the adsorption step. In an exemplary embodiment, the, adsorbents are selected to preferentially adsorb carbon oxides, nitrogen, methane, alcohols (especially methanol), ethers (especially dimethyl ether), and other oxygenates such as acetaldehyde. The remaining gas is rich in hydrogen and leaves the adsorber bed in the PSA product gas stream 187. When the adsorbent has reached its adsorption capacity, it is regenerated to prevent a breakthrough of contaminants into the PSA product gas stream 187. The process of regeneration of the PSA adsorbents is to depressure the adsorbent into a tail gas stream in line 186. The tail gas stream in line 186 comprises one or more by-product components from the second vapor stream in line 182. In an aspect, the tail gas stream in line 186 may be a gaseous waste stream.

As shown, from the PSA unit 185, the tail gas stream in line 186 comprising byproducts is separated from the PSA product gas stream in line 187, comprising hydrogen. In an exemplary embodiment, the hydrogen-rich PSA product stream in line 187 may be passed to the syngas pressure booster compressor 130 as a hydrogen stream. In an embodiment, the hydrogen stream in line 124 to the syngas pressure booster compressor 130 comprises the hydrogen-rich PSA product stream in line 187. The tail gas stream in line 186 is commonly used as fuel. In an embodiment, the tail gas stream in line 186 may be recycled to an additional conversion unit for conversion to syngas. This additional conversion unit may be a partial oxidation unit, steam reforming unit, autothermal reforming unit, or a dry reforming unit. In an exemplary embodiment, the tail gas stream in line 186 may be further processed in a dry reforming unit.

Turning back to the second gas-liquid separator 180, the second liquid stream in line 184 is withdrawn from the bottoms of the second gas-liquid separator 180 and passed to a third gas-liquid separator 190. The first liquid stream in line 154 may also be passed to the second gas-liquid separator 180. In an exemplary embodiment, the second liquid stream in line 184 may be combined with the first liquid stream in line 154 to provide a combined liquid stream in line 188 which is passed to the third gas-liquid separator 190. In the third gas-liquid separator 190, the first liquid stream in line 154 and the second liquid stream in line 184 are separated into a third vapor stream in line 192 and a third liquid stream in line 194. The third liquid stream in line 194 comprises crude methanol. Alternately, the third liquid stream in line 194 may be a crude methanol stream. The crude methanol stream may comprise at least 100 ppmw of carbon oxide and/or at least 100 ppmw C2+ oxygenates. The third vapor stream in line 192 comprises carbon oxides, hydrogen, and other byproducts that were dissolved in the combined liquid stream in line 188. This vapor stream is commonly used as a fuel. In an embodiment, the third vapor stream in line 192 may be combined with the tail gas stream in line 186 for further processing as described above. In an aspect, the third vapor stream in line 192 may be a gaseous waste stream.

The crude methanol comprises methanol, light ends, and heavier alcohols. As used and described herein, the term “crude methanol” or “crude oxygenate feedstock” may comprise methanol, ethanol, water, light ends, and fusel oil. The light ends may include ethers, ketones, aldehydes, and dissolved gases such as hydrogen, methane, carbon oxides, and nitrogen. The crude methanol comprises fusel oil. The fusel oil in the crude methanol typically includes higher molecular weight alcohols and is generally burned as a fuel in the methanol plant. The crude methanol comprising the fusel oil can be passed to the oxygenate conversion unit for the additional production of light olefins. The crude methanol may be passed to the oxygenate conversion unit or the MTO unit for feed. In accordance with the present disclosure, the crude methanol may be passed to the methanol purification unit (208) before passing to the oxygenate conversion unit or the MTO unit.

In accordance with an exemplary embodiment of the present disclosure, the crude methanol in line 196 may have a composition comprising carbon monoxide in a concentration from about 0 to about 1 wt %, carbon dioxide in a concentration from about 0.05 wt % to about 2 wt %, methane in a concentration from about from about 0.001 wt % to about 2 wt %, hydrogen in a concentration from about 0.05 wt % to about 2 wt %, oxygen in a concentration from about 0 to about 1 wt %, water in a concentration from about 5 wt % to about 18 wt %, nitrogen in a concentration from about 0 to about 1 wt %, methanol in a concentration from about 75 wt % to about 90 wt %, alcohols (other than methanol) in a concentration from about 0.01 to about 4 wt %, and ethers (specifically dimethyl ether) in a concentration from 0 to about 0.1 wt %.

The third liquid stream in line 194 may be passed to a crude methanol hold-up tank 195. A crude methanol stream in line 196 is withdrawn from the crude methanol hold-up tank 195.

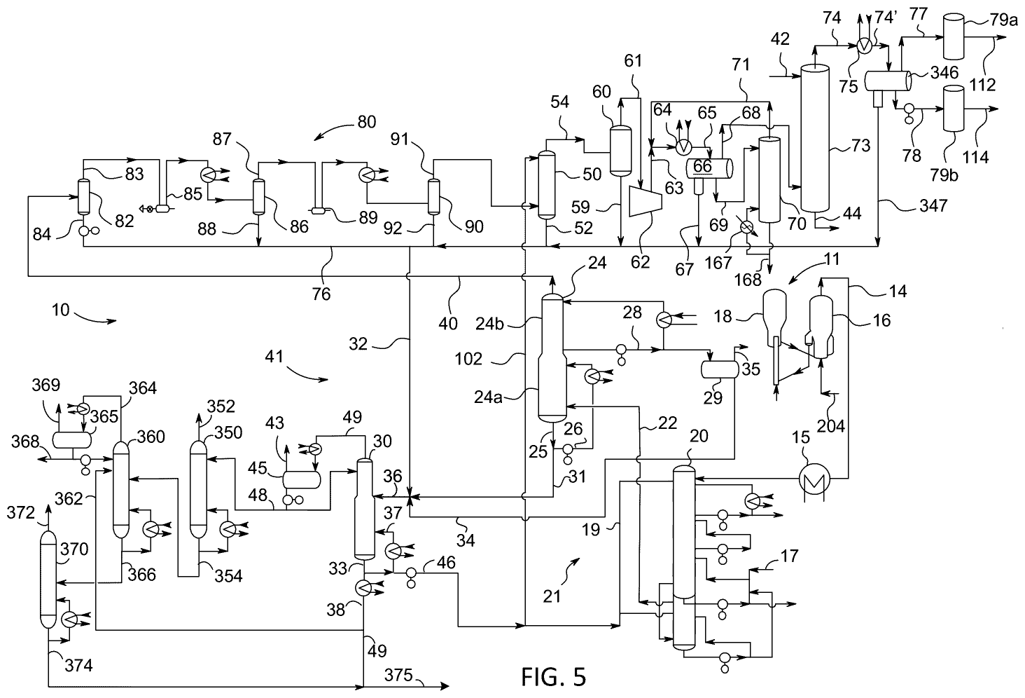

Conventionally, the crude methanol stream in line 196 is purified of the light gases and heavy oxygenates before it is charged to an MTO reactor, e.g., the oxygenate conversion reactor 16 of FIG. 5.

In accordance with an exemplary embodiment, the crude methanol stream in line 196 may be passed to the methanol purification section 208 comprising at least two distillation columns, a first distillation column 210 and a second distillation column 220. The crude methanol stream in line 196 is heat exchanged with a product stream in a heat exchanger 197 to provide a heated crude methanol stream in line 198. The heated crude methanol stream in line 198 may be passed to the first distillation column 210.

In the first distillation column 210, the light gas(es) are separated from the crude methanol in a first distillation column overhead stream in line 212. The light gases separated from the crude methanol stream include carbon monoxide, carbon dioxide, methane, hydrogen and dimethyl ether. The first distillation column overhead stream in line 212 is passed to a first overhead receiver 215 where the light gas(es) are separated in a first overhead receiver vapor stream in line 214. The first overhead receiver vapor stream in line 214 is commonly passed to a fuel system for use in the production of syngas or for heating fractionation columns. In an exemplary embodiment, first overhead receiver vapor stream in line 214 may be passed to a conversion unit comprising partial oxidation, steam reforming, autothermal reforming, or dry reforming to convert the light gasses to syn gas. From the first overhead receiver 215, an overhead receiver liquid stream is withdrawn in line 216 and passed to a top of the first distillation column 210. In an aspect, the first overhead receiver vapor stream in line 214 may be a gaseous waste stream.

A first distillation column bottoms stream comprising methanol in line 218 is withdrawn for further separation. The first distillation column bottoms stream in line 218 is separated into a first reboiling stream in line 218b and a first distillation column effluent stream in line 218a. The first reboil stream in line 218b is reboiled in a reboiler 219 before passing to the first distillation column bottom. In accordance with an exemplary embodiment, the first distillation column 210 is operated at a pressure from about 689 kPa (100 psia) to about 1379 kPa (200 psia). In accordance with another exemplary embodiment, the first distillation column is operated at a temperature of about 27° C. (80° F.) to about 177° C. (350° F.).

The first distillation column effluent stream in line 218a includes heavy oxygenates such as C2+ alcohols, ketones, aldehydes that should be removed from the crude methanol stream. Hence the first distillation column effluent stream in line 218a is further separated in a second distillation column 220. In the second distillation column 220, the first distillation column effluent stream in line 218a is separated into a second distillation column overhead stream in line 222 comprising methanol and a second distillation column bottoms stream in line 226. The overhead stream in line 222 is condensed for reflux to the column in line 228. From the heat exchanger 223, a condensed second distillation column overhead stream in line 224 is passed to a second overhead receiver 225. In the second overhead receiver 225, a portion of the condensed liquid of the second distillation column overhead stream in line 224 is refluxed to the second distillation column 220 via the second distillation column reflux stream in line 228. The remaining liquid from the second distillation column overhead is the methanol product stream in line 199 which is passed to the MTO reactor 16 in FIG. 5. In an aspect, the methanol product stream in line 199 is heat exchanged with the crude methanol stream in line 196 in the heat exchanger 197 to provide the heated crude methanol stream. The methanol product stream in line 199, after heat exchange with the crude methanol stream in line 196 in the heat exchanger 197 to provide a cooled methanol product stream in line 229, is passed to a methanol product hold-up tank 202. A methanol product stream in line 204 is withdrawn from the methanol product hold-up tank 202 for further processing as disclosed later in detail.

A second distillation column bottoms stream in line 226 is withdrawn from the column. The second distillation column bottoms stream in line 226 is separated into a second reboiling stream in line 226b and a second distillation column effluent stream in line 226a. The second reboiling stream in line 226b is reboiled in a reboiler 230 before passing to the second distillation column bottom section.

In accordance with an exemplary embodiment, the second distillation column is operating at a pressure from about 517 kPa (75 psia) to about 862 kPa (125 psia). In accordance with yet an exemplary embodiment, the second distillation column operates at a temperature of about 104° C. (220° F.) to about 149° C. (300° F.). The second distillation column effluent stream in line 226a will comprise heavy oxygenates and water, an aqueous oxygenate stream.

### Methanol to Olefins Conversion

FIG. 5 shows the olefinic feed preparation process and apparatus 10 of the present disclosure. The process 10 comprises an oxygenate conversion unit 41 which may be an MTO unit as shown in FIG. 5. The methanol charge stream is taken from the methanol product stream in line 204 of FIG. 4. The oxygenate conversion unit 41 comprises an oxygenate conversion section 11, a separation section 21, and a compression section 80. The methanol charge stream in line 204 may be processed in the oxygenate conversion section 11. The oxygenate conversion section 11 comprises an oxygenate conversion reactor 16 that reacts oxygenates such as methanol or dimethyl ether (DME) with fluidized catalyst. The separation section 21 comprises a quench tower 20, a product separator column 24, and a water stripper column 30. The methanol charge stream is fed via line 204 of FIG. 5 to the oxygenate conversion reactor 16. In an aspect, the methanol charge stream in line 204 may comprise one or more oxygenates including methanol, dimethyl ether, ethanol or combinations thereof. In the oxygenate conversion reactor 16, the charge stream is contacted with an MTO catalyst at MTO reaction conditions to convert methanol to olefins and water. The MTO catalyst may be in a fluidized state in the oxygenate conversion reactor 16.

Methanol is converted into light olefin products in a methanol to olefin (MTO) process. Molecular sieves such as microporous crystalline zeolite and non-zeolitic catalysts, particularly silicoaluminophosphates (SAPO), promote the conversion of oxygenates such as methanol to hydrocarbon mixtures, particularly hydrocarbon mixtures composed largely of light olefins. SAPO catalysts and their formulation are generally taught in U.S. Pat. Nos. 4,499,327A, 10,358,394 and 10,384,986. Light olefins produced from the MTO process are concentrated in ethylene and propylene but include C4-C6 olefins. In an embodiment, the MTO catalyst may be a SAPO catalyst.

The MTO reaction conditions include contact with a SAPO catalyst at a pressure between about 100 kPa (a) (14 psia) and about 700 kPa (a) (100 psia). The MTO reaction temperature should be between about 400° C. (750° F.) to about 510° C. (980° F.). A weight hourly space velocity (“WHSV”) in the oxygenate conversion reactor 16 is in the range of about 1 to about 15 hr⁻¹.

The MTO catalyst is separated from the product olefin stream after the MTO reaction. The hot vaporous reactor effluent stream in line 14 may be preliminarily cooled in a reactor effluent heat exchanger 15 to recover heat before it is passed to a quench tower 20. In the quench tower 20, the vaporous reactor effluent is desuperheated, neutralized of organic acids and clarified of catalyst fines by direct contact with a water stream supplied in line 19 which may be taken from a stripped water stream in line 21. A quenched reactor effluent stream in line 22 is discharged from the quench tower 20 and fed to a product separator column 24. The product separator column 24 may be in downstream communication with the oxygenate conversion reactor 16.

Product separator 24 comprises two sections a first, or lower, section 24a and a second, or upper section 24b for separating the reactor effluent stream into a product olefin stream in an overhead line 40, an intermediate liquid stream in an intermediate line 28 and a water stream in a bottoms line 25. A first, or lower, section 24a receives the quenched reactor effluent stream in line 22. In the lower section, most of the heat is removed from the quenched reactor effluent stream while partially condensing the water in the quenched reactor effluent stream to generate a product water stream in bottoms line 25 comprising a portion of the oxygenate byproducts in the quenched reactor effluent stream in line 22. A first portion of the product water stream in bottoms line 25 is pumped and cooled routed around in line 26 to the top of the first section 24a of the product separator 24 to cool the quenched reactor effluent stream in line 22. A second portion of the bottoms water stream in bottoms line 25 is taken in line 31 and passed to the water stripper column 30. A water return stream comprising oxygenate byproducts from the compression section 80 in return line 32 can also be passed to the water stripper column 30. The water stripper column 30 may be in downstream communication with the product separator column 24.

A vapor stream from the first section of the product separator 24 is passed to the second, or upper, section 24b of the product separator. An intermediate stream in line 28 comprising hydrocarbons, oxygenate byproducts, and water in liquid phase is withdrawn at a bottom of the upper section 24b. A portion of the intermediate stream in line 28 is cooled and passed as reflux to the top of the second section 24b of the product separator 24. The remainder of the intermediate stream in line 28 is passed to a coalescer 29 to separate a hydrocarbon overhead stream in line 35 from an aqueous stream in line 34 which is fed back to the product water stream and pumped to the water stripper column 30. An overhead product stream comprising olefins from the product separator column in line 40 is delivered to the compression section 80. In an aspect, the hydrocarbon overhead stream in line 35 may be a gaseous waste stream.

In accordance with an exemplary embodiment, the aqueous stream in line 34, the water return stream from the compression section 80 in return line 32, and the second portion of the bottoms water stream in line 31 are combined to provide a combined product water stream in line 36. The combined product water stream in line 36 is passed to the water stripper column 30. The combined product water stream in line 36 includes dilute oxygenates such as DME, methanol, acetaldehyde, acetone and methyl ethyl ketone (MEK). The water stripper column 30 separates or strips the oxygenates into a methanol and oxygenate rich stream in an overhead line 49 which is rich in both methanol and at least another oxygenate and a water rich stripper bottoms stream in a bottoms line 33. The water rich stripper bottoms stream in line 33 is split into a reboil stream in line 37 that is heated and returned to the column, a circulating stripped water stream in line 46 and a net stripper bottoms stream in line 38. The net stripper bottoms stream in line 38 may be split between a lean solvent stream in line 362 and a net stripped water stream in line 49. In one embodiment, the water stripper column 30 temperature may be about 115° C. (239° F.) to about 180° C. (356° F.) at the bottom of the water stripper column and the pressure may be about 75 kPa (g) (11 psig) to about 760 kPa (g) (110 psig) at the overhead of the water stripper column 30.

The overhead stream in line 49 is passed to an overhead receiver 45. Uncondensed light hydrocarbons can be purged from a receiver overhead line 43 while a methanol and oxygenate rich stream can be removed in a net overhead liquid line 48 and comprise methanol, DME, acetaldehyde, acetone and MEK. A portion of the methanol and oxygenate rich stream can be returned to the water stripper column 30 as reflux. The circulating stripped water stream in line 46 may be separated into a first water stream in line 19 and a second water stream in line 102. The first water stream in line 19 is fed to the quench column 20. The second water stream in line 102 may be passed to an absorber 50. In an aspect, the uncondensed light hydrocarbons stream in receiver overhead line 43 may be a gaseous waste stream.

The methanol and oxygenate rich stream in overhead liquid line 48 may be fed to the extractive distillation column 360 to separate methanol from at least one other oxygenate. However, the methanol and oxygenate rich stream in overhead liquid line 48 comprises DME which easily separates from methanol. Hence, the methanol and oxygenate rich stream in overhead liquid line 48 may be fed to a DME stripper column 350 to easily remove the DME. The DME stripper column 350 may be in downstream communication with the water stripper column 30. The DME stripper column 350 may separate or strip DME into a DME rich stream in an overhead line 352 and provide a DME lean, methanol and oxygenate rich stream in a bottoms line 354. The DME rich stream in the overhead line 352 may be recycled to the oxygenate conversion reactor 16 in the oxygenate conversion section 11 as reactant feed. A portion of the DME lean, methanol and oxygenate rich stream from the bottoms of the DME stripper column 350 may be reboiled and recycled to the DME stripper column 350. The DME lean, methanol and oxygenate rich stream in bottoms line 354 may be fed to the extractive distillation column 360. The extractive distillation column 360 may be in downstream communication with the water stripper column 30 and upstream of any communication with the product separator column 24 to assure that no inert oxygenates build up in the compression section without an avenue for return to the water stripper column 30. Additionally, in an embodiment, the extractive distillation column may be in downstream communication with the DME stripper column 350.

In one embodiment the DME stripper column 350 temperature may be about 85° C. (185° F.) to about 120° C. (248° F.) at the bottom of the DME striper column and the pressure may be about 75 kPa gauge (11 psig) to about 414 kPa (60 psig) at the top of the column. The DME stripper column 350 may utilize an overhead condenser and receiver separator in addition to or instead of the overhead condenser and receiver 45 for the water stripper column 30 to remove a light hydrocarbon purge. The DME stripper overhead can be recycled to oxygenate conversion section 11.

The DME lean, methanol and oxygenate rich stream in the net bottoms line 354 may be fed to an extractive distillation column 360 to separate methanol from at least one other hydrocarbon oxygenate and preferably all other hydrocarbon oxygenates. The lean solvent stream in line 362 may also be fed to the extractive distillation column 360 at a location, such as at the top quarter of the column, above a location, such as the middle quarter of the column, at which the DME lean, methanol and oxygenate rich stream in line 354 is fed to the column. The lean solvent stream may be provided in line 362 which may be taken from the water rich stream in the water stripper bottoms line 33.

The extractive distillation column 360 produces an oxygenate rich stream in an overhead line 364 comprising at least one oxygenate other than methanol such as, such as acetone, acetaldehyde, MEK, MIPK, DME, ethanol, C3 and C4 alcohols, acetaldehyde, acetic and formic acid, and a methanol rich extract stream in a bottoms line 366. A portion of the methanol rich stream in the bottoms line 366 may be reboiled and returned to the extractive distillation column 360. The oxygenate rich stream in the overhead line 364 may be cooled and partially condensed and fed to an extraction receiver 365. Uncondensed light hydrocarbons can be purged from a receiver overhead line 369 while an oxygenate rich stream can be removed in a net overhead liquid stream in line 368 and comprise acetaldehyde, acetone, and MEK. A portion of the oxygenate rich stream can be returned to the extractive distillation column 360 as reflux at a location above the location at which the lean solvent stream in line 362 is added to the extractive distillation column 360. The light hydrocarbon purge(s) may be fed to a fuel gas header. In an aspect, the uncondensed light hydrocarbons stream in receiver overhead line 369 may be a gaseous waste stream.

The extractive distillation column 360 may have operating conditions including a bottoms temperature in the range of about 75° C. (167° F.) to about 150° C. (302° F.) and an overhead pressure in the range of about 75 kPa (g) (11 psig) to about 200 kPa (g) (29 psig). The extractive distillation column 360 may be in downstream communication with the overhead line 49 of the water stripper column 30 and with a bottoms line 49 of the water stripper column 30.

The methanol and water rich stream in the net bottoms line 366 may be fed to a methanol stripper column 370 to separate a methanol rich stream in an overhead line 372 from a final water rich stream in a bottoms line 374. The methanol rich stream in the overhead line 372 may be recycled to the MTO reactor 16 without oxygenates that can otherwise build up in the process. A portion of the final water rich stream in the bottoms line 374 may be reboiled and recycled to the methanol stripper column 370. The final water rich stream in the net bottoms line 374 may be forwarded to water treatment in line 375 along with an unrecycled portion of the water rich stream in the net stripped water stream in the remaining bottoms line 49 from the water rich stripper bottoms stream in line 33.

The product olefin stream in the product overhead line 40 carries valuable olefinic products which must be recovered. The compression section 80 increases the pressure of the product olefin stream necessary for downstream processing such as used in conventional light olefin recovery units. The compression section 80 may comprise a first knock out drum 82 which separates the product olefin stream into a pressurized first olefin rich stream at a temperature of about 40° C. (104° F.) to about 60° C. (140° F.) and a pressure of about 193 kPa (g) (28 psig) to about 262 kPa (g) (38 psig) in an overhead line 83 and a first aqueous stream rich in oxygenates in a bottoms line 84. The olefin rich stream in the overhead line 83 may be fed to a compressor 85, cooled and directed to a second knockout drum 86. The aqueous stream in the bottoms line 84 is pumped via a manifold line 76 to the return line 32 which returns the water stream with the product water stream in the separator bottoms line 36 to the water stripper column 30.

The compression section 80 may comprise a second knock out drum 86 which separates the pressurized first olefin rich stream into a second pressurized olefin rich stream at a pressure of about 330 kPa (g) (48 psig) to about 400 kPa (g) (58 psig), and a temperature of about 27° C. (80° F.) to about 54° C. (130° F.) in an overhead line 87 and a second aqueous stream rich in oxygenates in a bottoms line 88. The second olefin rich stream in the overhead line 87 may be fed to a compressor 89, cooled and directed to a third knockout drum 90. The aqueous stream in the bottoms line 88 is pumped to the return line 32 via the manifold line 76 which returns the water stream with the product water stream in the separator bottoms line 36 to the water stripper column 30.

The compression section 80 may comprise a third knock out drum 90 which separates the pressurized second olefin rich stream into a third pressurized olefin rich stream in an overhead line 91 and a third aqueous stream rich in oxygenates in a bottoms line 92. The third olefin rich stream in the overhead line 91 may be fed to the oxygenate absorber column 50. The aqueous stream in the bottoms line 92 is passed to the return line 32 via manifold line 76 which returns the water stream with the product water stream in the separator bottoms line 36 to the water stripper column 30.

Types of suitable compressors may include centrifugal, positive displacement, piston, diaphragm, screw, and the like. In one embodiment, the compressors 85, 89 in the compression section 80 are centrifugal compressors. The final discharge pressure can be between about 1 MPa gauge (145 psig) and about 2 MPa gauge (290 psig). The compressor discharge may be cooled to about ambient temperatures using conventional heat transfer methods.

As illustrated in the FIG. 5 and according to a preferred embodiment, at least a portion of the compressed product stream via the overhead line 91 is contacted in the oxygenate absorber column 50 at effective conditions to absorb at least a quantity of effluent oxygenates with a cooled lean water stream in line 102 with no water taken directly from the product separator column 24 without prior removal of oxygenates. The contacting in the oxygenate absorber column 50 produces an oxygenate-lean olefin stream in the overhead line 54 and an oxygenate-rich water stream in a bottoms line 52 comprising a quantity of effluent oxygenates. The oxygenate absorber may have operating conditions including a bottoms temperature range of about 30° C. (86° F.) to about 60° C. (140° F.) and an overhead pressure range of about 700 kPa (g) (101 psig) to about 1 MPa(g) (145 psig).

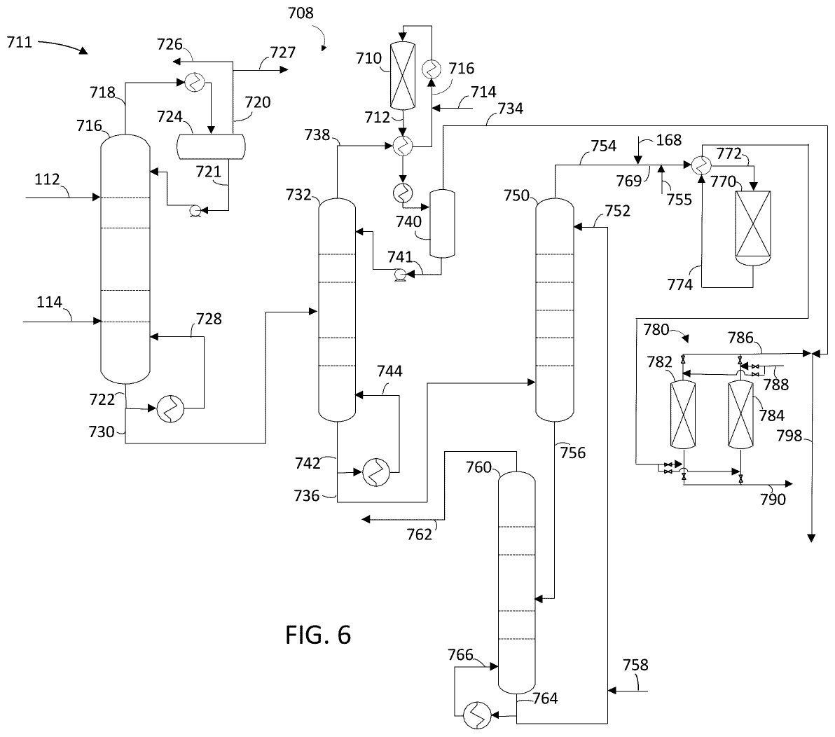

The oxygenate-rich olefin stream in the overhead line 54 may be fed to an absorber separator 60 in which a gaseous olefin stream is taken in an overhead line 61 to a third compressor 62 while water and oxygenates are taken in the bottoms line 59 to the manifold line 76. The gaseous olefin stream in line 61 is compressed in the third compressor 62, combined with the stripper overhead stream in line 71 via line 63, partially condensed by cooling in the heat exchanger 64 and fed in line 65 to a stripper separator 66. The stripper separator separates an aqueous stream including oxygenates in the boot in line 67 which feeds the manifold line 76, a light olefinic vapor stream in an overhead line 68 comprising C3− olefins and a heavy olefinic liquid stream comprising C4+ olefins in line 69. The heavy olefinic liquid stream in line 69 is stripped in a DME stripper column 70 to remove C3− and lighter vapors in a stripper overhead stream in line 71 from the heavy olefinic liquid stream in the stripper bottoms line 168. Most oxygenates will be stripped into the stripper overhead stream in line 71 and be separated after cooling upon recycle to the stripper separator 66. The bottom stream exiting the DME stripper column 70 may be sent through line 168 to selective hydrogenation reactor 770 in FIG. 6. This stream comprises mostly C4+ olefins but comprises diolefins that will deter the oligomerization catalyst requiring selective hydrogenation. The stripper separator may operate at a temperature of about 30° C. (86° F.) to about 60° C. (140° F.) and a pressure of about 1.7 MPa(g) (250 psig) to about 2.1 MPa(g) (300 psig). The light olefinic vapor stream in the overhead line 68 is scrubbed in a caustic scrubber column 73 by countercurrent contact with a caustic solution in line 42 to absorb acid gases such as carbon dioxide from the light olefinic vapor which exits the caustic scrubber 73 in an overhead line 74. The acid gas rich caustic solution exits scrubber 73 in line 44.

The scrubbed light olefinic vapor in overhead line 74 may be refrigerated by propylene refrigerant in a chiller 75 to liquefy part of the light olefinic stream in the chilled overhead line 74′ and separated in a drier separator 346 to provide an aqueous stream from a boot which is taken in boot line 347 to the manifold line 76, a vaporous light olefin stream comprising C2-hydrocarbons and gases in an overhead line 77 and a liquid light olefin stream in a bottoms line 78 comprising C3+ hydrocarbons. The vaporous light olefin stream in the overhead line 77 is dried in a drier 79a to provide a vaporous product olefin stream in line 112. The liquid light olefin stream in the bottoms line 78 is dried in a drier 79b to provide a liquid product olefin stream in line 114. The product olefin streams in lines 112 and 114 are processed in the oligomerization feed preparation section in FIG. 6.

FIG. 6 shows an embodiment of the oligomerization feed preparation section 711. In the oligomerization feed preparation section 711, the vaporous product olefin stream in line 112 of FIG. 5 is fed to a fractionation section 708. The vaporous product olefin stream in line 112 may comprise a C2 olefin stream and comprise predominantly ethylene. In an embodiment, a liquid product olefin stream in line 114 of FIG. 5 may be fed to the fractionation section 708. The liquid product olefin stream in line 114 may comprise a C3 olefin stream and comprise predominantly propylene. In the fractionation section, the vaporous product olefin stream in line 112 and/or the liquid product olefin stream in line 114 are fractionated to provide a light gas stream in line 720 and a rich olefin stream in line 722.

In an embodiment, the vaporous product olefin stream in line 112 may be fed to a demethanizer fractionation column 716. The vaporous product olefin stream in line 112 may be fed to the top half of the demethanizer fractionation column 716. In an embodiment, a liquid product olefin stream in line 114 may be fed to the demethanizer fractionation column 716. The liquid product olefin stream in line 112 may be fed to the bottom half of the demethanizer fractionation column 716. The vaporous product olefin stream and the liquid product olefin stream may be fractionated in the demethanizer fractionation column 716 together.

The vaporous product olefin stream and/or the liquid product olefin are fractionated preferably together in the demethanizer fractionation column 716 to provide an overhead light gas stream in an overhead light gas line 718 and a bottom rich olefin stream in a bottoms line 722. The overhead light gas stream in line 718 may comprise light gases of methane and lighter gases such as carbon monoxide, carbon dioxide, methane, nitrogen and hydrogen. Essentially all of the carbon monoxide will exit in the overhead light gas stream in line 718. The overhead light gas stream in line 718 is cooled and fed to a demethanizer receiver 724. Condensed light gases are refluxed from the demethanizer receiver 724 to column 716 in a reflux line 721 while the light gas stream is taken in a net overhead line 720. A reactor purge gas stream in line 726 can be taken from the light gas stream to the MTO reactor and a fuel gas stream can be taken in line 727. In an aspect, the fuel gas stream in line 727 may be a gaseous waste stream.

The rich olefin stream comprising C2+ olefins, typically C2-C6 olefins, in the demethanizer bottoms line 722 may be split into a reboil stream in line 728 which is reboiled and returned to the column and a net rich olefin stream in a net bottoms line 730. The demethanizer bottoms temperature may be about 10° C. (50° F.) to about 54° C. (100° F.) and a pressure of about 2.4 MPa(g) (350 psig) to about 3.5 MPa(g) (500 psig).

The rich olefin stream comprises appreciable levels of dienes, acetylenes, dimethyl ether and other oxygenates which are all harmful to the oligomerization catalyst. In an embodiment, the rich olefin stream may be further fractionated to prepare the ethylene and the propylene separately. If ethylene is routed to the selective hydrogenation reactor 770, the acetylene may fully saturate making it inert in an oligomerization reactor. Consequently, the net rich olefin stream in line 730 may be further fractionated in a deethanizer column 732.

The net rich olefin stream in line 730 is deethanized by fractionation in the deethanizer column 732 to provide an ethylene stream in a net overhead line 734 and a fractionated rich olefin stream in a deethanized net bottoms line 736. The deethanizer column 730 may be operated at a bottoms temperature of about 43° C. (110° F.) to about 104° C. (220° F.) and an overhead pressure of about 2.1 kPa (g) (300 psig) to about 3.5 kPa (g) (500 psig).

An ethylene overhead stream in an overhead line 738 may be heated by heat exchange with a concentrated ethylene stream in line 712 and combined with a hydrogen stream from line 714 to provide a combined ethylene overhead stream in combine line 716, further heated and charged to an acetylene conversion reactor 710. In the acetylene conversion reactor 710, acetylenes are converted to ethylene over an acetylene conversion catalyst in the presence of hydrogen thereby producing a concentrated ethylene stream in line 712. The concentrated ethylene stream in line 712 is condensed by heat exchange with the ethylene overhead stream in the overhead line 738 and further condensed. The further condensed concentrated ethylene stream is separated in a deethanizer receiver 740 to provide the ethylene stream of vapor phase in the net overhead line 734 and a condensed liquid stream in a reflux line 741. Condensate from the deethanizer receiver 740 may be refluxed back to the deethanizer column 732 in the reflux line 741 from a bottom of the deethanizer receiver 740. The deethanized stream in the bottoms line 742 may be split between a reboil stream in line 744 which is reboiled and returned boiling to the deethanizer column 732 to provide heating requirements. The acetylene conversion catalyst may be a palladium and silver on aluminum oxide catalyst. The acetylene conversion conditions may include a pressure of about 1.4 MPa(g) (200 psig) to about 2.8 MPa(g) (400 psig) and a temperature of about 37° C. (100° F.) to about 93° C. (200° F.).

The fractionated rich olefin stream in the net bottoms line 736 may contain oxygenates such as dimethyl ether and other oxygenates in concentration that would poison selective hydrogenation catalyst. Hence, the rich olefin stream in line 736 is routed to a water wash column 750 to absorb oxygenates from the fractionated rich olefin stream.

In the water wash column 750, a water wash stream from a DME wash water stripper column 760 is routed in an absorbent line 752 to a top third of the water wash column and countercurrently contacted with the fractionated rich olefin stream in the net bottoms line 736 fed to a bottom third of the water wash column. Countercurrent contact of the fractionated rich olefin stream and the water wash stream effects absorption of the oxygenates including DME from the fractionated rich olefin stream into the water wash stream. Absorption produces a washed olefin stream in an overhead line 754 and an oxygenate rich water wash stream in a bottoms line 756. The washed olefin stream in the overhead line 754 has a total oxygenate concentration of no more than 1000 wppm which is acceptable for the selective hydrogenation catalyst in the selective hydrogenation reactor 770. Suitably, the washed olefin stream in the overhead line 754 has a total oxygenate concentration of no more than 500 wppm to moderate the adsorbent bed size required in the oxygenate removal unit 780. Preferably, the washed olefin stream in the overhead line 754 has a total oxygenate concentration of no more than 200 wppm. In a preferred embodiment, the washed olefin stream in the overhead line 754 has a total oxygenate concentration of less than 50 wppm. The water wash column 750 may be operated at a bottoms temperature of about 26° C. (50° F.) to about 66° C. (150° F.) and an overhead pressure of about 2.4 MPa(g) (350 psig) to about 3.2 MPa(g) (465 psig).

The oxygenate rich water wash stream in the bottoms line 756 is fed to the DME wash water stripper column 760 to be stripped of DME and other oxygenates. In the DME wash water stripper column 760, DME and oxygenates are stripped from the oxygenate rich water wash stream to produce a DME stream in line 762 which also contains other oxygenates which can be recycled to the MTO reactor (not shown). A stripped water wash stream is produced in a bottoms line 764. A reboil stream in line 766 is taken from the stripped water wash stream in the bottoms line 764 reboiled and transported back to the DME wash water stripper column 760. The water wash stream in the absorbent line 752 is taken from the stripped water wash stream in the bottoms line 764 and recycled to the water wash column 750 perhaps after supplementation with make-up water in line 758.

The washed olefin stream in line 754 may be oligomerized in an oligomerization reactor perhaps in liquid phase. However, the washed olefin stream in the water wash overhead line 754 comprising C3 to C6 olefins also contains diolefins that could cause cross-link polymerization in the oligomerization reactor. Therefore, it may be selectively hydrogenated to convert diolefins to mono-olefins. The C4+ olefins in the DME stripped line 168 from FIG. 5 may also contain diolefins and can accordingly also benefit from selective hydrogenation prior to oligomerization Hence, the washed olefin stream in the water wash overhead line 754 may be combined with the heavy olefin stream in line 168 to provide a combined olefin stream in a combine line 769. The combined olefin stream in the combine line 769 may be mixed with hydrogen from line 755 heated perhaps by heat exchange with the mono-olefin stream in line 774 and charged to the selective hydrogenation reactor 770 in the selective hydrogenation charge line 772. In the selective hydrogenation reactor 770, diolefins and residual acetylenes are converted to mono-olefins to provide the mono-olefin stream in line 774. Selective hydrogenation effects just minimal hydrogenation of mono-olefins to paraffins.

The selective hydrogenation reactor 770 is normally operated at relatively mild hydrogenation conditions. These conditions will normally result in the hydrocarbons being present as liquid phase materials. The reactants will normally be maintained under the minimum pressure sufficient to maintain the reactants as liquid phase hydrocarbons. Suitable operating pressures include about 2.3 MPa(g) (330 psig) to about 3.1 MPa(g) (450 psig). A relatively moderate temperature between about 20° C. (68° F.) and about 100° C. (212° F.) is typically employed. The liquid hourly space velocity of the reactants through the selective hydrogenation catalyst should be above about 1.0 hr⁻¹ and about 35.0 hr⁻¹. To avoid the undesired saturation of a significant amount mono-olefinic hydrocarbons, the mole ratio of hydrogen to diolefinic hydrocarbons in the selective hydrogenation charge line 772 entering the bed of selective hydrogenation catalyst is maintained between 0.75:1 and 1.8:1.

Any suitable catalyst which is capable of selectively hydrogenating diolefins in a naphtha range stream may be used. Suitable catalysts include, but are not limited to a catalyst comprising copper and at least one other metal such as titanium, vanadium, chrome, manganese, cobalt, nickel, zinc, molybdenum, palladium, and cadmium or mixtures thereof. The metals are preferably supported on inorganic oxide supports such as silica and alumina, for example. The mono-olefin stream may exit the reactor in line 774 with a greater concentration of ethylene and a smaller concentration of acetylenes and diolefins than in the selective hydrogenation charge stream in line 772. The mono-olefin stream in line 774 may comprise an acetylene and diolefin concentration of no more than about 50 to about 80 wppm.

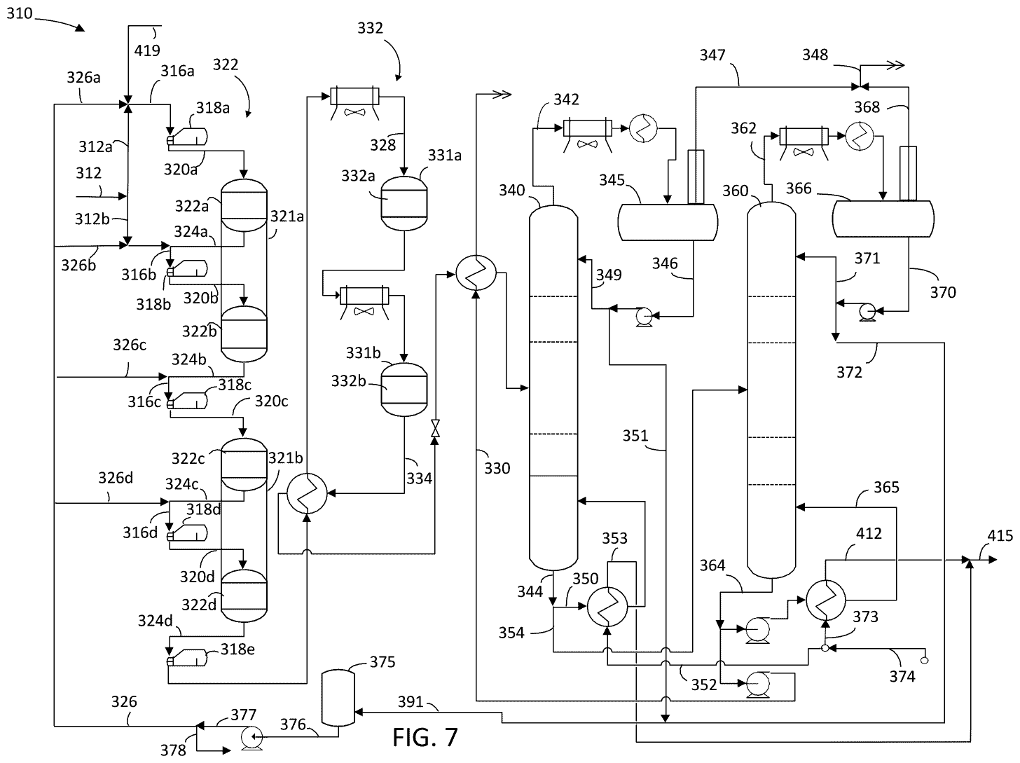

The mono-olefin stream in line 774 may be oligomerized in a downstream oligomerization reactor perhaps in liquid phase. However, the mono-olefin stream still has a large concentration of oxygenates that could cause undesirable catalyst deactivation in the oligomerization reactor 322 in FIG. 7. Accordingly, the selectively hydrogenated stream in line 774 may be transported to an oxygenate removal unit 780 to adsorb residual oxygenates including DME and water. The oxygenate removal unit 780 may comprise one or more adsorbent vessels 782, 784, so one adsorbent vessel 782 or 784 can be charged with the mono-olefin stream in line 774 by appropriate valve control to adsorb oxygenates therefrom while the other adsorbent vessel 784 or 782 can be fed with a regenerant stream from line 788 by appropriate valve control to undergo regeneration. The mono-olefin stream in line 774 may flow upwardly in the adsorbent vessel 782, 784, but downstream is also suitable. Three adsorbent vessels may be used in the oxygenate removal unit to ensure that one adsorbent vessel is always operating in adsorption mode. A deoxygenated olefin stream can be recovered from the oxygenate removal unit 780 in line 786. An oxygenated regenerant stream may be recovered from the oxygenate removal unit 780 in line 790. The deoxygenated olefin stream in line 786 comprises C3 to C6 olefins and not more than 1 wppm oxygenate including DME and water.

When an adsorbent vessel 782, 784 requires regeneration, it can be taken off-stream with the selectively hydrogenated stream in line 774 and contacted with a heated vaporous regenerant from line 788 through appropriate valve control in a direction counter to the normal flow of the olefinic selectively hydrogenated stream. The regenerant may be a clean inert gas such as nitrogen, hydrogen, natural gas and light paraffins such as propane, butanes and pentanes. The regenerant can fully restore the capacity of the adsorbent in the regenerated vessel 782, 784. The spent regenerant can leave the oxygenate removal unit 780 in a spent regenerant line 790. The oxygenate removal unit may be operated at an inlet temperature of about 26° C. (50° F.) to about 66° C. (150° F.) and an inlet pressure of about 2.3 MPa(g) (330 psig) to about 3 MPa(g) (435 psig). The adsorbent in the oxygenate removal unit 780 may be a large pore molecular sieve such as 13X molecular sieve.

The deoxygenated olefin stream in line 786 may provide an oligomerization charge stream in line 798 that can be charged to the downstream oligomerization reactor. Alternatively or cumulatively, the concentrated ethylene stream in line 734 may be charged to the oligomerization reactor in the oligomerization charge stream in line 798.

### Oligomerization and Hydrogenation

Olefin oligomerization is a process that can oligomerize smaller olefins into larger olefins. More specifically, it can convert light olefins including oligomerized olefins into olefins with higher carbon number, including gasoline, jet, and diesel range products. The olefins can be saturated for use as transportation fuels.

The oligomerization section 310 is illustrated in FIG. 7. A charge olefin stream and a recycle olefin stream are oligomerized with a first-stage oligomerization catalyst to produce a first-stage oligomerized olefin stream. The first-stage oligomerized olefin stream is oligomerized with a second-stage oligomerization catalyst to provide a second-stage oligomerized stream. A dealkanizer column can be used to remove light alkanes that would be inert and accumulate in a recycle loop. The second-stage oligomer product stream is hydrogenated to produce fuels.

Turning to the oligomerization section 310 of FIG. 7, a charge olefin stream in line 312 is provided to the oligomerization section 310. The charge olefin stream may comprise substantial ethylene and propylene. The charge olefin stream may predominantly comprise ethylene and/or propylene. In an aspect, the charge olefin stream may comprise at least 80 mol % ethylene and/or propylene. The charge olefin stream in line 312 may be styled a light olefin stream. Additional olefinic species with carbon numbers ranging from C3 to C6 can be expected in the charge stream. The light olefin stream may be provided by the dehydration of ethanol or provided from a MTO unit. The charge olefin stream may be at a temperature of about 35° C. (95° F.) to about 150° C. (302° F.), preferably about 80° C. (176° F.) to about 100° C. (212° F.), and a pressure of about 3.5 MPa(g) (500 psig), preferably about 5.6 MPa(g) (800 psig) to about 8.4 MPa(g) (1200 psig). In an embodiment, the charge olefin stream in line 312 may comprise or be taken from the oligomerization charge stream in line 798 in FIG. 6.

The charge olefin stream may be initially contacted with a first-stage oligomerization catalyst to oligomerize the ethylene and propylene to oligomers and then contacted with a second oligomerization catalyst to oligomerize unconverted ethylene and propylene from the first-stage oligomerization. Alternatively, the olefin stream may be initially contacted with a second stage oligomerization catalyst to oligomerize ethylene and propylene, and then be contacted with the first-stage oligomerization catalyst to oligomerize the oligomerized ethylene and propylene.

The oligomerization reaction generates a large exotherm. For example, dimerization of ethylene can generate 612 kcal/kg (1100 BTU/lb) of heat. Consequently, this large exotherm must be managed. Accordingly, the charge olefin stream in line 312 may be split into multiple olefin streams. In FIG. 7, the charge olefin stream is split into two separate streams: a first charge olefin stream in a first charge olefin line 312a and a second charge olefin stream in a second charge olefin line 312b. More or less separate multiple olefin streams may be used. Up to six charge olefin streams are readily contemplated. The charge olefin stream in line 312 may be split into equal aliquot multiple olefin streams. Alternatively, the charge olefin stream in line 312 may be split into unequal streams. For example, the charge olefin stream may be split into streams of descending flow rates in which a charge olefin stream to a preceding reactor has a larger flow rate than a charge olefin stream to a subsequent reactor. In an embodiment, the charge olefin stream is split into two streams of equal flow rates, each comprising 50 vol % of the charge olefin stream. In another embodiment, the first charge olefin stream in the first charge olefin line 312a may comprise about 70 to about 90 vol % of the charge olefin stream and the second charge olefin stream in the second olefin line 312b may comprise about 10 to about 30 vol % of the charge olefin stream.

To manage the exotherm, the charge olefin stream may be diluted with a diluent stream to provide a diluted olefin stream to absorb the exotherm. The diluent stream in a diluent line 419 may comprise paraffins. The diluent stream in the diluent line 419 may be added to the first charge olefin stream in the first charge olefin line 312a before it is charged to the first-stage oligomerization reactor 322. Preferably, the diluent stream is added to the first charge olefin stream in line 312a after the split of the charge olefin stream in line 312 into multiple olefin streams to provide a first diluted olefin charge stream in line 316a, so the diluent stream passes through all of the first-stage oligomerization reactions. Alternatively, the diluent stream may also be split into multiple streams with each diluent stream added to a corresponding charge olefin stream. The diluent stream may have a mass flow rate of about 1 to about 8 times and preferably about 3 to about 6 times the mass flow rate of the charge olefin stream in the charge olefin line 312.

A recycle olefin stream in line 326 comprising C4 to C8 olefins may be mixed with the charge olefin stream and oligomerized in the first-stage oligomerization reactor 322. In an embodiment, the recycle olefin stream in line 326 is split into a plurality of recycle olefin streams 326a-326d. A recycle olefin stream in a first recycle olefin line 326a may be mixed with the first charge olefins stream in line 312a and charged to the first-stage oligomerization reactor 322. In a further embodiment, the first recycle olefin stream in the first recycle olefin line 326a is mixed with the first charge olefin stream in line 312a and the diluent stream in line 419 to provide a diluted first charge olefin stream in line 316a.

The first diluted charge olefin stream may comprise no more than 35 wt % olefins, suitably no more than 30 wt % olefins and preferably no more than 20 wt % olefins. In an embodiment, the first diluted olefin stream comprises about 10 to about 30 wt % C2 to C8 olefins. The first diluted olefin stream may comprise no more than 30 wt % ethylene, suitably no more than 25 wt % ethylene and preferably no more than 20 wt % ethylene. In an embodiment, the first diluted charge olefin stream comprises about 10 to about 20 wt % propylene. The first diluted charge olefin stream may comprise no more than 30 wt % propylene, suitably no more than 25 wt % propylene and preferably no more than 20 wt % propylene. In an embodiment, the first diluted charge olefin stream comprises about 10 to about 20 wt % propylene.

The first-stage oligomerization reactor 322 may comprise a series of first-stage oligomerization catalyst beds 322a, 322b, 322c and 322d each for charging with an olefin charge stream 312a, 312b, 312c, and 312d, respectively. The first-stage oligomerization 322 reactor preferably contains four fixed first-stage oligomerization catalyst beds 322a, 322b, 322c and 322d. It is also contemplated that each first-stage oligomerization catalyst bed 322a, 322b, 322c and 322d may be in a dedicated first-stage oligomerization reactor or multiple first-stage oligomerization catalyst beds may be in two or more separate first-stage oligomerization reactor vessels. Up to six first-stage oligomerization catalyst beds are readily contemplated. In FIG. 7, two first stage oligomerization reactor vessels 321a and 321b are utilized.

A parallel first-stage oligomerization reactor may be used when the first-stage oligomerization reactor 322 has deactivated during which the first-stage oligomerization reactor 322 is regenerated in situ by combustion of coke from the catalyst. In another embodiment, each first-stage oligomerization reactor may comprise a lead reactor, a lag reactor and a spare reactor to facilitate regeneration. Only two reactor vessels 321a, 321b are shown in FIG. 7.

The diluted first charge olefin stream in line 316a may be cooled in a first charge cooler 318a to provide a cooled diluted first charge olefin stream in line 320a and charged to a first bed 322a of first-stage oligomerization catalyst in the first, first-stage oligomerization reactor vessel 321a of the first-stage oligomerization reactor 322. The cooled diluted first charge olefin stream in line 320a may be charged at a temperature of about 180° C. (356° F.) to about 260° C. (500° F.) and a pressure of about 3.5 MPa(g) (500 psig) to about 8.4 MPa(g) (1200 psig). The charge cooler 318a may comprise a steam generator.

The diluted first charge olefin stream may be charged to the first, first-stage catalyst bed 322a in line 320a preferably in a down flow operation. However, upflow operation may be suitable. As oligomerization of ethylene, propylene and recycle olefins occurs in the first, first-stage oligomerization catalyst bed 322a, an exotherm is generated due to the highly exothermic nature of the olefin oligomerization reaction. Oligomerization of the first charge olefin stream produces a first oligomerized effluent stream in a first oligomerized effluent line 324a at an elevated outlet temperature despite the cooling and dilution. The elevated outlet temperature is limited to between 150° C. (302° F.) and about 250° C. (482° F.).

The second charge olefin stream in line 312b may be mixed with a second recycle olefin stream in a second recycle olefin line 326b and with the first oligomerized effluent stream in the first oligomerized effluent line 324a removed from the first, first-stage oligomerization catalyst bed 322a in the first, first-stage reactor 321a to provide a mixed second charge olefin stream in line 316b. The first oligomerized effluent stream in line 324a includes the diluent stream from diluent line 419 added to the first olefin charge stream in line 312a. The second charge olefin stream may comprise no more than 35 wt % C2 to C8 olefins, suitably no more than 25 wt % C2 to C8 olefins and preferably no more than 20 wt % ethylene. The second charge olefin stream may comprise no more than 30 wt % ethylene, suitably no more than 25 wt % ethylene and preferably no more than 20 wt % ethylene. The second charge olefin stream may comprise no more than 30 wt % propylene, suitably no more than 25 wt % propylene and preferably no more than 20 wt % propylene. The second charge olefin stream in line 316b may be cooled in a second charge cooler 318b which may be located externally to the first, first-stage oligomerization reactor 321a to provide a cooled second charge olefin stream in line 320b and charged to a second bed 322b of first-stage oligomerization catalyst in the first, first-stage oligomerization reactor 321a. The charge cooler 318b may comprise a steam generator.

The second charge olefin stream in line 320b may be charged at a temperature of about 180° C. (356° F.) to about 230° C. (446° F.) and a pressure of about 3.5 MPa(g) (500 psig) to about 8.4 MPa(g) (1200 psig). The second charge olefin stream will include diluent and olefins from the first oligomerized stream. The olefins from the first oligomerized stream will oligomerize in the second catalyst bed 322b. Oligomerization of ethylene, propylene, recycle olefins and oligomers in the second olefin stream in the second bed 322b of first-stage oligomerization catalyst produces a second oligomerized olefin effluent stream in a second oligomerized effluent line 324b at an elevated outlet temperature. The elevated outlet temperature may be limited to between 30° C. (54° F.) and about 50° C. (90° F.) above the inlet temperature to the catalyst bed 322b.

The second oligomerized effluent stream in line 324b removed from the second, first-stage oligomerization catalyst bed 322b in the first, first-stage reactor vessel 321a may be mixed with a third recycle olefin stream in a third recycle olefin line 326c to provide a first recycle olefin charge stream in line 316c. None of the charge olefin stream in line 312 is directly added to the first recycle olefin charge stream in line 316c. Alternatively, a portion of the charge olefin stream in line 312 may be charged with the second oligomerized effluent stream with the first recycle olefin charge stream in line 316c. The second oligomerized effluent stream in line 324b includes the diluent stream from diluent line 419 added to the first charge olefin stream in line 312a. The first recycle olefin charge stream may comprise no more than 30 wt % ethylene, suitably no more than 25 wt % ethylene and preferably no more than 20 wt % ethylene. The first recycle olefin charge stream may comprise no more than 30 wt % propylene, suitably no more than 25 wt % propylene and preferably no more than 20 wt % propylene. The first recycle olefin charge stream may comprise no more than 30 wt % C2 to C8 olefins, suitably no more than 25 wt % C2 to C8 olefins and preferably no more than 20 wt % C2 to C8 olefins. The first recycle olefin charge stream in line 316c may be cooled in a third charge cooler 318c which may be located externally to the oligomerization reactor 322 to provide a cooled first recycle olefin charge stream in line 320c and charged to a third bed 322c of first-stage oligomerization catalyst in the first-stage oligomerization reactor 322. In an embodiment, the third bed 322c of first-stage oligomerization catalyst is provided in a second, first-stage oligomerization reactor vessel 321b. The charge cooler 318c may comprise a steam generator.

The cooled first recycle olefin charge stream in line 320c may be charged at a temperature of about 180° C. (356° F.) to about 230° C. (446° F.) and a pressure of about 3.5 MPa(g) (500 psig) to about 8.4 MPa(g) (1200 psig). The first recycle olefin charge stream will include diluent and olefins from the second oligomerized olefin stream and the third recycle olefin stream. The olefins will oligomerize in the third catalyst bed 322c. Oligomerization of ethylene and propylene and oligomerization of oligomers in the first recycle olefin charge stream in the third bed 322c of first-stage oligomerization catalyst produces a third oligomerized effluent stream in a third oligomerized effluent line 324c at an elevated outlet temperature. In an embodiment, the third oligomerized effluent stream is a penultimate oligomerized effluent stream and the third oligomerized effluent line 324c is a penultimate oligomerized effluent line 324c. The elevated outlet temperature is limited to between 30° C. (54° F.) and about 50° C. (90° F.) above the inlet temperature to the catalyst bed 322c.

The third oligomerized effluent stream in line 324c removed from the second, first-stage oligomerization reactor vessel 321b of the first-stage oligomerization reactor 322 may be mixed with the fourth recycle olefin stream in line 326d to provide a second recycle olefin charge stream in line 316d. The third oligomerized effluent stream in line 324c includes the diluent stream from diluent line 419 added to the first olefin stream in line 312a. None of the charge olefin stream in line 312 is directly added to the second recycle olefin charge stream in line 316d. In an embodiment, the third oligomerized effluent stream in line 324c may also be mixed with an olefin charge stream from the olefin charge line 322 and be oligomerized therewith. The second recycle olefin charge stream may comprise no more than 35 wt % C2 to C8 olefins, suitably no more than 30 wt % C2 to C8 olefins and preferably no more than 25 wt % C2 to C8 olefins. The second recycle olefin charge stream may comprise no more than 30 wt % ethylene, suitably no more than 25 wt % ethylene and preferably no more than 20 wt % ethylene. The second recycle olefin charge stream may comprise no more than 30 wt % propylene, suitably no more than 25 wt % propylene and preferably no more than 20 wt % propylene. The second recycle olefin charge stream in line 316d may be cooled in a fourth charge cooler 318d which may be located externally to the second vessel 321b of the first-stage oligomerization reactor 322 to provide a cooled second recycle olefin charge stream in line 320d and charged to a fourth bed 322d of first-stage oligomerization catalyst in the second vessel of the first-stage oligomerization reactor 322. The charge cooler 318d may comprise a steam generator.

The cooled second recycle olefin charge stream in line 320d may be charged at a temperature of about 180° C. (356° F.) to about 230° C. (446° F.) and a pressure of about 3.5 MPa(g) (500 psig) to about 8.4 MPa(g) (1200 psig). The cooled second recycle olefin charge stream in line 320d will include diluent and olefins from the third or penultimate oligomerized effluent stream and C4-C8 olefins from the fourth recycle olefin stream. The olefins will oligomerize over the fourth catalyst bed 322d. Oligomerization of ethylene and propylene in the second recycle olefin charge stream in the fourth bed 322d of first-stage oligomerization catalyst produces a fourth oligomerized stream in a fourth oligomerized effluent line 324d at an elevated outlet temperature. The elevated outlet temperature is limited to between 30° C. (54° F.) and about 50° C. (90° F.) above the inlet temperature to the catalyst bed 322d.

The fourth oligomerized effluent stream in line 324d exits the second reactor vessel 321b of the first-stage oligomerization reactor 322. In an embodiment, the fourth oligomerized effluent stream in line 324d is a last oligomerized effluent stream, and the fourth oligomerized effluent line 324d is a last oligomerized effluent line 324d.

The first-stage oligomerization reaction takes place predominantly in the liquid phase or in a mixed liquid and gas phase at a WHSV 0.5 to 10 hr⁻¹ on an olefin basis We have found that across the first-stage oligomerization catalyst beds, typically 30-50 wt % ethylene in the olefin stream converts to higher olefins. The ethylene will initially dimerize over the catalyst to butenes. A predominance of the propylene and butenes in the olefins stream charged to a first-stage oligomerization catalyst bed is oligomerized. In an embodiment, at least 99 mol % of propylene and butenes in the olefins stream are oligomerized.

The first-stage oligomerization catalyst may include a zeolitic catalyst. The first-stage oligomerization catalyst may be considered a solid acid catalyst. The zeolite may comprise between about 5 and about 95 wt % of the catalyst, for example between about 5 and about 85 wt %. Suitable zeolites include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. Three-letter codes indicating a zeotype are as defined by the Structure Commission of the International Zeolite Association and are maintained at http://www.iza-structure.org/databases. UZM-8 is as described in U.S. Pat. No. 6,756,030. In a preferred aspect, the first-stage oligomerization catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the first-stage oligomerization catalyst comprising a zeolite having a ten-ring pore structure may comprise a uni-dimensional pore structure. A uni-dimensional pore structure indicates zeolites containing non-intersecting pores that are substantially parallel to one of the axes of the crystal. The pores preferably extend through the zeolite crystal. Suitable examples of zeolites having a ten-ring uni-dimensional pore structure may include MTT. In a further aspect, the first-stage oligomerization catalyst comprises an MTT zeolite.

The first-stage oligomerization catalyst may be formed by combining the zeolite with a binder, and then forming the catalyst into pellets. The pellets may optionally be treated with a phosphorus reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the catalyst binder utilized in the present invention is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark VERSAL. A preferred alumina is available from Sasol North America Alumina Product Group under the trademark CATAPAL. This material is an extremely high purity alpha-alumina monohydrate (pseudo-boehmite) which after calcination at a high temperature has been shown to yield a high purity gamma-alumina.

A suitable first-stage oligomerization catalyst is prepared by mixing proportionate volumes of zeolite and alumina to achieve the desired zeolite-to-alumina ratio. In an embodiment, the MTT content may about 5 to about 85, for example about 20 to about 82 wt % MTT zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

In one exemplary embodiment, an MTT-type zeolite catalyst disposed on a high purity pseudo boehmite alumina substrate in a ratio of about 90/10 to about 20/80 and preferably between about 20/80 and about 50/50 is provided in a catalyst bed or more in the first-stage oligomerization reactor 322.

The first-stage oligomerization catalyst can be regenerated upon deactivation. Suitable regeneration conditions include subjecting the first-stage oligomerization catalyst, for example, in situ, to hot air at about 400 to about 500° C. for 3 hours. To facilitate regeneration without downtime, a swing bed arrangement may be employed with an alternative first-stage oligomerization reactor. A regeneration gas stream may be admitted to the first-stage oligomerization reactor 322 requiring regeneration. The regeneration gas may comprise air with an increased or decreased concentration of oxygen. Activity and selectivity of the regenerated catalyst is comparable to fresh catalyst.

The zeolite catalyst is advantageous as a first-stage oligomerization catalyst. The zeolitic catalyst has relatively low sensitivity towards oxygenates contamination. Consequently, a smaller degree of removal of oxygenates is required of olefinic feed in line 312 if produced from an ethanol dehydration process.

The last first-stage oligomerized stream in the last first-stage oligomerized effluent line 324d has an increased concentration of ethylene and propylene oligomers compared to the charge olefin stream in line 312. The last first-stage oligomerized stream in the last first-stage oligomerized effluent line 324d is cooled by steam generation in a steam generator 318e or by other heat exchange and further cooled by heat exchange against a second stage oligomerized stream in line 334 and perhaps further cooled such as by an air cooler to provide a charge first-stage oligomerized stream and charged to a second-stage oligomerization reactor 332 in a second-stage oligomerization charge line 328. To achieve the most desirable olefin product, the second-stage oligomerization reactor 332 is operated at a temperature from about 80° C. (176° F.) to about 220° C. (428° F.). The second-stage oligomerization reactor 332 is run at a pressure from about 2.1 MPa(g) (300 psig) to about 7.6 MPa(g) (1100 psig), and more preferably from about 3.5 MPa(g) (500 psig) to about 6.9 MPa(g) (1000 psig).

The second-stage oligomerization reactor 332 may be in downstream communication with the first-stage oligomerization reactor 322. The second-stage oligomerization reactor 332 preferably operates in a down flow operation. However, upflow operation may be suitable. The second-stage oligomerization charge stream is contacted with the second-stage oligomerization catalyst causing the unconverted ethylene and propylene from the first-stage oligomerization reactor 322 to dimerize and trimerize while higher olefins also dimerize, trimerize and tetramerize to provide distillate range olefins. With regard to the second-stage oligomerization reactor 332, process conditions are selected to produce a higher percentage of jet range olefins which, when hydrogenated in a subsequent step, result in a desirable jet-range hydrocarbon product. A predominance of the unconverted ethylene from the first-stage oligomerization reactor 322 is dimerized, trimerized and tetramerized. In an embodiment, at least 99 wt % of ethylene in the second-stage oligomerization charge stream is converted to mostly butenes.

The second-stage oligomerization reactor 332 may comprise a first reactor vessel 331a comprising a first bed 332a of second-stage oligomerization catalyst and a second reactor vessel 331b comprising a second bed 332b of second-stage oligomerization catalyst. A first, second-stage oligomerized stream is discharged from the first, second-stage reactor vessel 331a, cooled and charged to the second, second-stage reactor vessel 331b. A second-stage oligomerized stream with an increased average carbon number greater than the charge first-stage oligomerized stream in line 328 exits the second-stage oligomerization reactor 332 in line 334.

The second-stage oligomerization catalyst is preferably an amorphous silica-alumina base with a metal from either Group VIII and/or Group VIB in the periodic table using Chemical Abstracts Service notations. In an aspect, the catalyst has a Group VIII metal promoted with a Group VIB metal. Typically, the silica and alumina will only be in the base, so the silica-to-alumina ratio will be the same for the catalyst as for the base. The metals can either be impregnated onto or ion exchanged with the silica-alumina base. Co-mulling is also contemplated. Catalysts for the present invention may have a Low Temperature Acidity Ratio of at least about 0.15, suitably of about 0.2, and preferably greater than about 0.25, as determined by Ammonia Temperature Programmed Desorption (Ammonia TPD) as described hereinafter. Additionally, a suitable catalyst will have a surface area of between about 50 and about 400 m²/g as determined by nitrogen BET.

The preferred second-stage oligomerization catalyst comprises an amorphous silica-alumina support. One of the components of the catalyst support utilized in the present invention is alumina. The alumina may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A particularly preferred alumina is available from Sasol North America Alumina Product Group under the trademark CATAPAL. This material is an extremely high purity alpha-alumina monohydrate (pseudo-boehmite) which after calcination at a high temperature has been shown to yield a high purity gamma-alumina. Another component of the catalyst support is an amorphous silica-alumina. A suitable silica-alumina with a silica-to-alumina ratio of 2.6 is available from CCIC, a subsidiary of JGC, Japan.

Another component utilized in the preparation of the second-stage oligomerization catalyst utilized in the present invention is a surfactant. The surfactant is preferably admixed with the hereinabove described alumina and the silica-alumina powders. The resulting admixture of surfactant, alumina and silica-alumina is then formed, dried and calcined as hereinafter described. The calcination effectively removes by combustion the organic components of the surfactant but only after the surfactant has dutifully performed its function in accordance with the present invention. Any suitable surfactant may be utilized in accordance with the present invention. A preferred surfactant is a surfactant selected from a series of commercial surfactants sold under the trademark “Antarox” by Solvay S.A. The “Antarox” surfactants are generally characterized as modified linear aliphatic polyethers and are low-foaming biodegradable detergents and wetting agents.

A suitable silica-alumina mixture is prepared by mixing proportionate volumes silica-alumina and alumina to achieve the desired silica-to-alumina ratio. In an embodiment, about 75 to about 99 wt-% amorphous silica-alumina with a silica-to-alumina ratio of 2.6 and about 10 to about 20 wt-% alumina powder will provide a suitable support. In an embodiment, other ratios of amorphous silica-alumina to alumina may be suitable.

Any convenient method may be used to incorporate a surfactant with the silica-alumina and alumina mixture. The surfactant is preferably admixed during the admixture and formation of the alumina and silica-alumina. A preferred method is to admix an aqueous solution of the surfactant with the blend of alumina and silica-alumina before the final formation of the support. It is preferred that the surfactant be present in the paste or dough in an amount from about 0.01 to about 10 wt-% based on the weight of the alumina and silica-alumina.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried.

The paste or dough may be prepared in the form of shaped particulates, with the preferred method being to extrude the dough mixture of alumina, silica-alumina, surfactant and water through a die having openings therein of desired size and shape, after which the extruded matter is broken into extrudates of desired length and dried. A further step of calcination may be employed to give added strength to the extrudate. Generally, calcination is conducted in a stream of dry air at a temperature from about 260° C. (500° F.) to about 815° C. (1500° F.).

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

Typical characteristics of the amorphous silica-alumina supports utilized herein are a total pore volume, average pore diameter and surface area large enough to provide substantial space and area to deposit the active metal components. The total pore volume of the support, as measured by conventional mercury porosimeter methods, is usually about 0.2 to about 2.0 cc/gram, preferably about 0.25 to about 1.0 cc/gram and most preferably about 0.3 to about 0.9 cc/gram. Ordinarily, the amount of pore volume of the support in pores of diameter greater than 100 angstroms is less than about 0.1 cc/gram, preferably less than 0.08 cc/gram, and most preferably less than about 0.05 cc/gram. Surface area, as measured by the B.E.T. method, is typically above 50 m²/gram, e.g., above about 200 m²/gram, preferably at least 250 m²/gram., and most preferably about 300 m²/gram to about 400 m²/gram.

To prepare the second-stage oligomerization catalyst, the support material is compounded, as by a single impregnation or multiple impregnations of a calcined amorphous refractory oxide support particles, with one or more precursors of at least one metal component from Group VIII or VIB of the periodic table. The Group VIII metal, preferably nickel, should be present in a concentration of about 0.5 to about 15 wt-% and the Group VIB metal, preferably tungsten, should be present in a concentration of about 0 to about 12 wt-%. The impregnation may be accomplished by any method known in the art, as for example, by spray impregnation wherein a solution containing the metal precursors in dissolved form is sprayed onto the support particles. Another method is the multi-dip procedure wherein the support material is repeatedly contacted with the impregnating solution with or without intermittent drying. Yet other methods involve soaking the support in a large volume of the impregnation solution or circulating the support therein, and yet one more method is the pore volume or pore saturation technique wherein support particles are introduced into an impregnation solution of volume just sufficient to fill the pores of the support. On occasion, the pore saturation technique may be modified, so as to utilize an impregnation solution having a volume between about 10 percent less and about 10 percent more than that which will just fill the pores.

If the active metal precursors are incorporated by impregnation, a subsequent or second calcination at elevated temperatures, as for example, between 399° C. (750° F.) and 760° C. (1400° F.), converts the metals to their respective oxide forms. In some cases, calcinations may follow each impregnation of individual active metals. A subsequent calcination yields a catalyst containing the active metals in their respective oxide forms.

A preferred second-stage oligomerization catalyst of the present invention has an amorphous silica-alumina base impregnated with about 0.5 to about 15 wt-% nickel in the form of 3.175 mm (0.125 inch) extrudates and a density of about 0.45 to about 0.65 g/ml. It is also contemplated that metals can be incorporated onto the support by other methods such as ion-exchange and co-mulling.

The second-stage oligomerization catalyst can be regenerated upon deactivation. Suitable regeneration conditions include subjecting the catalyst, for example, in situ, to hot air at about 400 to about 500° C. for 3 hours. To facilitate regeneration without downtime, a swing bed arrangement may be employed with an alternative second-stage oligomerization reactor. The regeneration gas may comprise air with an increased or decreased concentration of oxygen. Activity and selectivity of the regenerated catalyst is comparable to fresh catalyst.

Second-stage oligomerization reactions are also exothermic in nature. The last oligomerized olefin stream in line 324d includes the diluent stream from diluent line 419 added to the first olefin stream in line 312a and carried through the first-stage oligomerization catalyst beds 322a-322d. The diluent stream is then transported into the second-stage oligomerization reactor 332 in line 328 to absorb the exotherm in the second-stage oligomerization reactor. A dedicated diluent line to second-stage oligomerization reactor 332 is also contemplated for prompt control of exotherm rise or to cool down the second-stage oligomerization reactor 332 independently.

When the oligomerization reaction is performed according to the above-noted process conditions, a C2 olefin conversion of greater than or equal to about 95% is achieved, or greater than or equal to 97%. The resulting second-stage oligomerized stream in line 334 includes a plurality of olefin products that are distillate range hydrocarbons.

An oligomerized olefin stream in line 334 with an increased C8+ olefin concentration compared to the charge first-stage oligomerization stream in line 328 is heat exchanged with the first-stage oligomerized stream in line 324d, let down in pressure, subsequently heat exchanged with an olefin splitter bottoms stream in line 330 and fed to a dealkanizer column 340. The oligomerized olefin stream in line 334 is at a temperature from about 160° C. (320° F.) to about 190° C. (374° F.) and a pressure of about 3.9 MPa(g) (550 psig) to about 7 Mpa(g) (1000 psig). The olefin splitter bottoms stream in line 330 may be transported to a hydrogenation section.

We have found that light alkanes such as ethane and/or propane are generated in the first-stage oligomerization reactor 322 and/or the second-stage oligomerization reactor 332 which must be removed from the second-stage oligomerized stream for fuels production particularly to facilitate light olefin recycle to the first-stage oligomerization reactor 322. Light alkanes are inert and would accumulate in the recycle loop. Hence, the second-stage oligomerized stream in line 334 is dealkanized by fractionation in a dealkanizer column 340 to provide a light alkane stream and a dealkanized stream. In an embodiment, the light alkane stream is an ethane stream in which case the dealkanizer column 340 is a deethanizer column. In another embodiment, the light alkane stream is a propane stream in which case the dealkanizer column 340 is a depropanizer column. The alkane stream may also be a mixture of ethane and propane. The alkane stream may be used as fuel for providing heating duty in the process 310. The alkane stream may also be converted to syngas via steam reforming, partial oxidation, autothermal reforming, or dry reforming, and syngas can be recycled to the methanol synthesis unit.

In the dealkanizer column 340, light alkanes such as C3− and suitably C2− hydrocarbons, are separated perhaps in an alkane overhead stream in an overhead line 342 of FIG. 7 from perhaps a dealkanized bottoms stream in a bottoms line 344 comprising C4+ and suitably C3+ hydrocarbons. The dealkanizer column 340 may be operated at a bottoms temperature of about 177° C. (350° F.) to about 302° C. (575° F.) and an overhead pressure of about 207 kPa (gauge) (30 psig) to about 690 kPa (gauge) (100 psig) if operated as a deethanizer column. The dealkanizer column 340 may be operated at a bottoms temperature of about 194° C. (381° F.) to about 333° C. (630° F.) and an overhead pressure of about 207 kPa (gauge) (30 psig) to about 1.14 MPa(g) (165 psig) if operated as a depropanizer column.

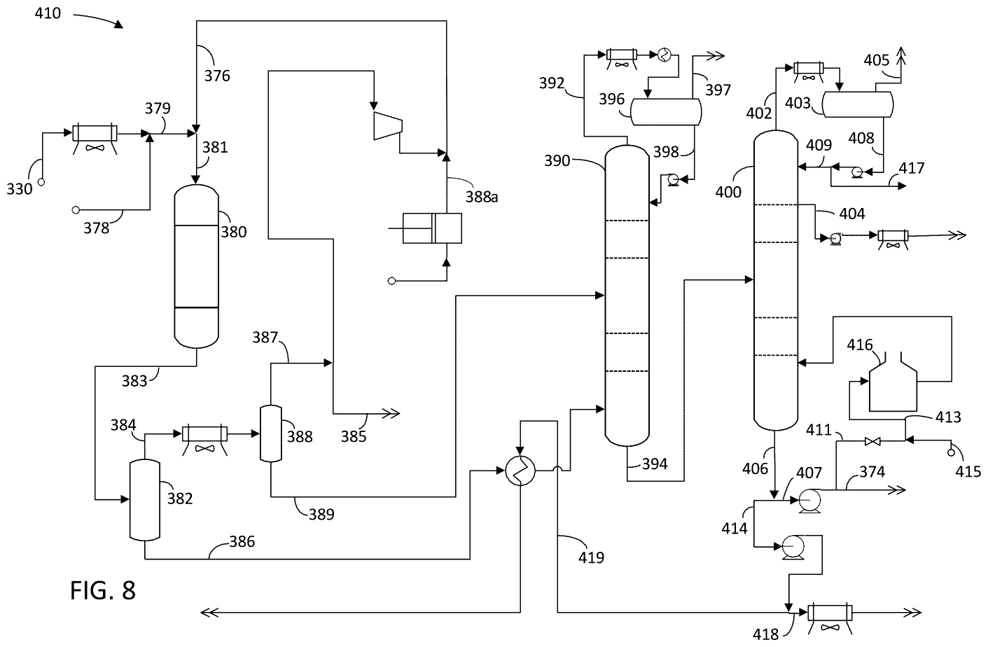

The alkane overhead stream in the overhead line 342 may be cooled and separated in a dealkanizer receiver 345 to provide a first net vapor stream in line 347. A dealkanizer overhead liquid stream is taken in line 346 from the dealkanizer receiver 345. Condensate from the dealkanizer receiver 345 may be refluxed back to the dealkanizer column 340 in a reflux line 349. In an embodiment, some of the condensate from the dealkanizer overhead liquid stream in line 346 may be taken as recycle in line 351 to the first stage oligomerization reactor in lines 372 and 326. The dealkanized stream perhaps in the bottoms line 344 may be split between a reboil stream in line 350 which is reboiled by heat exchange with the third circulating jet fractionator bottoms stream in line 352 perhaps taken from the second circulating jet fractionator bottoms line 374, and a net bottoms stream in line 354, which is fed directly to an olefin splitter column 360 perhaps without heating. The cooled third circulating jet fractionator bottoms stream in line 353 is returned back to the hydrogenation section 410 in FIG. 8 to be reboiled and fed back to the jet fractionation column 400. The reboiled bottom stream in line 350 may be returned boiling to the dealkanizer column 340 to provide heating requirements. In another embodiment, feed to dealkanizer column 340 is not preheated by olefin splitter bottoms stream in line 330, but the feed to the olefins splitter column 360 in the net bottoms line 354 would be preheated by olefin splitter bottoms stream.

The dealkanized stream in the dealkanizer net bottoms line 354 is split by fractionation in an olefin splitter column 360 into a light olefin stream perhaps in an olefin splitter overhead line 362 and a heavy olefin stream perhaps in an olefin splitter bottoms line 364. The olefin splitter overhead stream may be cooled to about 66° C. (150° F.) to about 93° C. (200° F.) and a resulting condensate portion refluxed from an olefin splitter receiver 366 back to the olefin splitter column 360. The vapor from the olefin splitter receiver 366 may be chilled to provide the second net vapor stream in line 368. The first net vapor stream in line 347 and the second net vapor stream in line 368 may be combined into a combined net vapor stream in line 348. The combined net vapor stream in line 348 is commonly sent to fuel gas to provide fuel for fractionation reboilers or syngas generation. In an exemplary embodiment of the present disclosure, the combined net vapor stream in line 348 is sent to further processing to create syngas via partial oxidation, steam reforming, or autothermal reforming. In an aspect, the combined net vapor stream in line 348 may be a gaseous waste stream.

The light olefin condensate from a bottom of the olefin splitter receiver in line 370 may be split between a reflux stream that is refluxed back to the column in line 371 and a light olefin recycle stream in a recycle line 372 that may be recycled to the first-stage oligomerization reactor 322 or alternatively to the second-stage oligomerization reactor 332. The light olefin stream in line 372 may comprise about 1 to about 15 wt % of the light olefin stream in line 370. The light olefin stream in line 372 may comprise about 40 to about 80 wt % C4-C8 olefins. In an embodiment, the light olefin stream in line 372 may be combined with the recycle stream in line 351 and sent to a surge drum 375 in a recycle line 391. A combined recycle olefin stream is taken from the bottom of the surge drum 375 in line 376. In an aspect, the combined recycle olefin stream is pumped and a pumped recycle olefin stream in line 377 is passed to the first-stage oligomerization reactor 322 to oligomerize the C4-C8 olefins. In an embodiment, a portion of the recycle olefin stream may be taken as drag stream in a drag line 378 and sent to a hydrogenation section 410 in FIG. 8. The remaining recycle olefin stream is taken in line 326 and recycled to the first-stage oligomerization reactor 322. In an exemplary embodiment, the light olefin drag stream in line 378 may range between about 2% and about 10%, preferably between about 3% and about 4% of the recycle olefin stream in line 326.

The heavy olefin stream in the splitter bottoms line 364 may be split between a reboil stream in a splitter reboil line 365 that is reboiled by heat exchange with the fourth circulating jet fractionator bottoms stream in line 373, perhaps taken from the second circulating jet fractionator bottoms stream in line 374 from FIG. 8 and fed back to the olefin splitter column 360. The cooled fourth circulating jet fractionator bottoms stream in line 412 is returned back to the hydrogenation section 410 in FIG. 8 to be reboiled and fed back to the jet fractionation column 400. In an embodiment, the cooled fourth circulating jet fractionator bottoms stream in line 412 is combined with the cooled third circulating jet fractionator bottoms stream in line 353 to provide a combined cooled circulating jet fractionator bottoms stream in line 415 which is passed back to the hydrogenation section 410 in FIG. 8. The heavy olefin stream in the net bottoms line 330 is cooled by heat exchange with the second-stage oligomerized stream in line 334 before it is transported to the hydroprocessing section 410, as shown in FIG. 8. The heavy olefin stream comprises C8+ olefins that, once cooled, can be transported to the hydrogenation section 410.

### Hydrogenation

Turning to the hydrogenation section 410 in FIG. 8, the heavy olefin stream in the net olefin splitter bottoms line 330 from FIG. 7 comprising distillate-range C9+ oligomerized olefins may be hydrogenated to saturate the olefinic bonds in a hydrogenation reactor 380 to provide fuels. This step is performed to ensure the product motor fuel meets or exceeds the thermal oxidation requirements specified in ASTM D7566-20 for Alcohol to Jet Synthesized Paraffinic Kerosene (ATJ-SPK). Additionally, hydrogenating the oligomerized heavy olefins will provide the paraffin stream that may be used as the diluent stream in line 419. The heavy olefin stream in line 330 may be cooled to produce steam and be combined with the light olefin drag stream in line 378 also from FIG. 7 to produce a combined olefin stream in line 379. The combined olefin stream in line 379 may also be combined with a hydrogen stream in line 376 to provide a combined hydrogenation charge stream in line 381 which is cooled and charged to the hydrogenation reactor 380 at 125° C. (257° F.) to about 204° C. (400° F.) and 3.5 MPa(g) (500 psig) to about 6.9 MPa(g) (1000 psig). An excess of hydrogen may be employed to ensure complete saturation. The H2:olefin ratio at the inlet to the hydrogenation reactor 380 may be from about 1.5 to about 2.5.

Hydrogenation is typically performed using a conventional hydrogenation or hydrotreating catalyst, and can include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to oxides such as silica, alumina, titania, calcium carbonate, barium sulfate, and carbons. The catalyst support can be in the form of powder, granules, pellets, or the like.

In an exemplary embodiment, hydrogenation is performed in the hydrogenation reactor 380 that includes a platinum-on-alumina catalyst, for example about 0.5 wt % to about 0.9 wt % platinum-on-alumina catalyst. In another embodiment, the hydrogenation catalyst comprises about 5 to about 30 wt % nickel catalyst. The hydrogenation reactor 380 converts the olefins into a paraffin product having the same carbon number distribution as the olefins, thereby forming distillate-range paraffins suitable for use as jet and diesel fuel.

The hydrogenated heavy stream discharged from the hydrogenation reactor 380 in line 383 may be separated in a hot separator 382 which provides a hydrocarbon split. In the hot separator 382, the hydrogenated heavy stream is separated into a hot hydrogenated vapor stream in an overhead line 384 and a hot hydrogenated liquid stream in the hot separator bottoms line 386. The hydrogenated heavy liquid stream in the bottoms line 386 may be heated by heat exchange with the diluent stream in line 419 before the diluent stream is recycled to the first-stage oligomerization reactor 322 in FIG. 7. The heated hydrogenated heavy liquid stream in the hot bottoms line 386 may be fed to a stripping column 390. The hot separator may be operated at a temperature of about 204° C. (400° F.) to about 343° C. (650° F.) and a pressure of 3.5 MPa(g) (500 psig) to about 6.9 MPa(g) (1000 psig).

The hot hydrogenated vapor stream in the hot overhead line 384 may be cooled and fed to a cold separator 388. The cold separator separates the cooled hot hydrogenated vapor stream in the hot overhead line 384 into a cold vapor hydrogenated stream in a cold overhead line 387 and a cold heavy hydrogenated liquid stream in a cold bottoms line 389. A purge stream in a purge line 385 may be taken from the cold vapor hydrogenated stream in the cold overhead line 387 and the remainder may be compressed and combined with make-up hydrogen in line 388a to provide the hydrogen stream in line 376. The cold hydrogenated heavy liquid stream in the bottoms line 389 may be fed to the stripping column 390 at a feed location above that for the hot hydrogenated heavy liquid stream in the hot separator bottoms line 386. The cold separator may be operated at a temperature of about 32° C. (90° F.) to about 71° C. (150° F.) and a pressure of about 500 psig to about 1000 psig, or preferably between 3.7 MPa(g) (540 psig) and about 4.5 MPa(g) (650 psig).

The stripping column 390 may be a flash stripper to remove light gases from the hot hydrogenated liquid stream in the hot bottoms line 386 and the cold hydrogenated liquid stream in the cold bottoms line 389. Both of these streams can be combined and charged to the stripping column 390 or they can be charged separately as shown. The stripping column 390 removes residual light gases from the liquid hydrogenated streams to provide a stripper overhead stream in a stripper overhead line 392 and a stripped bottom stream in a stripper bottoms line 394. The stripper overhead stream in the stripper overhead line 392 is cooled and separated in a stripper receiver 396 to provide a stripper net overhead vapor stream in a stripper receiver overhead line 397 and a stripper overhead liquid stream in line 398 which is refluxed to the column. The stripper net overhead vapor stream in line 397 is commonly sent to fuel to provide fuel for the fractionation reboil heater 416 or to provide heat to produce syngas feed. In an exemplary embodiment of the present disclosure, the stripper net overhead vapor stream in line 397 is processed for conversion into syngas in a partial oxidation unit, steam reforming unit, or autothermal reforming unit. The stripping column 390 may be operated at a bottoms temperature of about 232° C. (450° F.) to about 316° C. (700° F.) and an overhead pressure of about 207 kPa (g) (30 psig) to about 689 kPa (g) (100 psig). In an aspect, the stripper net overhead vapor stream in line 397 may be a gaseous waste stream.

After undergoing stripping to remove volatiles in the stripping column 390, the stripped bottoms stream in the stripper bottoms line 394 may be fed to the jet fractionation column 400 without further heating. Alternatively, the stripping column 390 may be omitted upstream of the jet fractionation column 400. In the jet fractionation column 400, the stripped bottoms stream may be separated into a jet fractionation overhead stream in line 402, a jet fractionation sidedraw stream in line 404 from a side of the jet fractionation column 400, and a jet fractionation bottoms stream in line 406. The jet fractionation column 400 may be operated at a bottoms temperature of about 288° C. (550° F.) to about 416° C. (780° F.) and an overhead pressure of about 35 kPa (g) (5 psig) to about 350 kPa (g) (50 psig).

The jet fractionation overhead stream in line 402 may be cooled. Some or all of the resulting condensate is refluxed from a jet fractionation receiver 408 back to the jet fractionation column 400 in a jet fractionator reflux line 409, a portion of the condensate is taken in a jet fractionator net overhead liquid stream in line 417, and a jet fractionator net vapor stream comprising C8-hydrocarbons is taken in a receiver overhead line 405 from the jet fractionation receiver 408. Most of the hydrocarbons in the jet fractionator net vapor stream in the receiver overhead line 405 are lighter hydrocarbons and are commonly used to fuel the reboil heater 416 for the jet fractionation column 400. In an exemplary embodiment of the present disclosure, the jet fractionator net vapor stream in line 405 is processed for conversion into syngas in a partial oxidation unit, steam reforming unit, or autothermal reforming unit. The jet fractionator net overhead liquid stream in line 417 comprises naphtha and light components and is commonly used as fuel or can be used as a gasoline blending component. In an exemplary embodiment of the present disclosure, the jet fractionator net overhead liquid stream in line 417 can be processed for conversion into syngas in a partial oxidation unit, steam reforming unit, or autothermal reforming unit. In an aspect, the jet fractionator net vapor stream in line 405 may be a gaseous waste stream.

The jet fractionator sidedraw stream in line 404 comprises kerosene range C8-C18 hydrocarbons and may be cooled and taken as a jet fuel product meeting applicable SPK standards. In an alternative embodiment, the green jet stream may be taken from the jet fractionator net overhead stream in line 417 from the jet fractionation receiver 403. If the green jet stream is taken from the jet fractionator net overhead stream in line 417, it may have to be further stripped to remove light ends. In such an embodiment, no jet fractionator sidedraw stream in line 404 would be taken from the jet fractionator 400. In an aspect, the jet fractionator net overhead stream in line 417 may be a liquid waste stream comprising naphtha.

The jet fractionator bottoms stream in line 406 may be split between a first circulating jet fractionator bottoms stream in line 407 and a recycle jet fractionator bottoms stream in line 414. The first circulating jet fractionator bottoms stream in line 407 may be split into a second circulating jet fractionator bottoms stream in line 374 and a bypass circulating jet fractionator bottoms stream in line 411. As shown in FIG. 7, the second circulating jet fractionator bottoms stream in line 374 may be split between a third circulating jet fractionator bottoms stream in line 352 and a fourth circulating jet fractionator bottoms stream in line 373 to provide reboiler heat to the dealkanizer column 340 and the olefin splitter column 360, respectively. The cooled third circulating jet fractionator bottoms stream in line 353 and cooled fourth circulating jet fractionator bottoms stream in line 412 are combined into a first combined cooled circulating jet fractionator bottoms stream in line 415. The first combined cooled circulating jet fractionator bottoms stream in line 415 and the bypass circulating jet fractionator bottoms stream in line 411 are combined to provide a second combined cooled circulating jet fractionator bottoms stream in line 413, which is passed to the jet fractionator reboiler heater 416 and fed back to the jet fractionation column 400.

The recycle jet fractionator bottoms stream in line 414 is split between a diesel product stream in line 418 and a diluent stream in line 419. The diluent stream in line 419 may be cooled by heat exchange with the hot hydrogenated heavy liquid stream in the hot separator bottoms line 386 and recycled back to be mixed with the olefin stream in line 312 in the oligomerization section 310 in FIG. 7, preferably the first charge olefin stream in line 312a, to provide the first diluted olefin charge stream in line 316a to absorb the exotherm in the oligomerization reactor 322. The diluent stream in the diluent line 419 is paraffinic, so it will be inert to the oligomerization and hydrogenation reactions to which it may be subject. The diesel product stream in the diesel product line 418 may be cooled and fed to the diesel pool. The diesel stream will meet ASTM D975 standards for diesel. Due to the highly selective nature of this process to produce jet fuel, it is possible that the operator may not need to produce a separate diesel product. In an exemplary embodiment of the present disclosure, the diesel product stream in line 418 may be converted to syngas feed in a partial oxidation process, steam cracking process, or autothermal reforming process. In an aspect, the diesel product stream in line 418 may be a liquid waste stream.

Starting with ethylene and/or propylene, the disclosed process can efficiently produce green jet fuel and green diesel fuel that meets applicable fuel requirements while managing exothermic heat generation. Carbon recovery in the process can exceed 95%. Both the jet fuel stream in the side line 404 and the diesel product stream in line 418 can be cooled and fed to their respective fuel pools.

The applicants have found that carbon recovery can be increased if the waste stream and/or less desirable products are recycled back to the methanol synthesis unit. In order for the methanol synthesis unit to consume these streams, they need to be converted to syngas via partial oxidation, steam reforming, autothermal reforming, or dry reforming, where applicable. If partial oxidation or autothermal reforming is selected to produce the syngas, oxygen must also be provided, and additional steam could be extracted from the reactor for use elsewhere in the facility. If steam reforming is selected to produce the syngas from ethane/propane, it will also need steam feeds which could be fully supplied by the process and energy input which could be partially supplied by steam from the process. Dry reforming requires energy input and carbon dioxide.

Various gaseous waste streams have been disclosed above as potential streams that can be recycled to the methanol synthesis unit. The gaseous waste streams may include the tail gas stream in line 186, the third vapor stream in line 192, and first overhead receiver vapor stream in line 214 in the methanol synthesis unit 101 in FIG. 4. From the MTO unit 41 in FIG. 5, the hydrocarbon overhead stream in line 35, the uncondensed light hydrocarbons stream in receiver overhead line 43 and the uncondensed light hydrocarbons stream in receiver overhead line 369 can be employed as a gaseous waste stream. From the oligomerization feed preparation section 711, the fuel gas stream in line 727 can be employed as a gaseous waste stream. From the oligomerization section 310 in FIG. 7, the combined net vapor stream in line 348 can be utilized as a gaseous waste stream. From the hydrogenation section 410 of FIG. 8, the stripper net overhead vapor stream in line 397 and the jet fractionator net vapor stream in line 405 can be used as a gaseous waste stream. Other byproduct waste liquid streams from the hydrogenation section 410 may optionally be converted to syngas, depending on the processing objectives of the operator. These waste liquid streams include the jet fractionator net overhead liquid stream comprising naphtha in line 417 and the diesel product stream in line 418. While partial oxidation and steam reforming can convert all of these streams to syngas, certain streams are more suitable for one type of conversion over another.

### Partial Oxidation

A syngas stream comprising carbon oxides and hydrogen may be produced by partially oxidizing hydrocarbon-rich streams with limited amounts of oxygen. Suitable candidate streams for partial oxidation are the combined net vapor stream in line 348 in the oligomerization section 310 of FIG. 7; and the stripper net overhead vapor stream in line 397, the jet fractionator net vapor stream in line 405, optionally the jet fractionator net overhead liquid stream in line 417, and optionally the diesel product stream in line 418 in the hydrogenation section 410 of FIG. 8.

In an exemplary embodiment of the present disclosure, the partial oxidation reaction can be arranged to provide a portion of the energy required to reboil one or more fractionation columns. FIG. 9 shows the partial oxidation of waste streams to provide heat to the jet fractionation column 400 in FIG. 8 of the oligomerization unit hydrogenation section 410. Partial oxidation of waste streams is integrated with circulating jet fractionator bottoms. In the partial oxidation reactor, oxygen is introduced at levels less than the stoichiometric requirement for complete combustion of the waste stream. Partial oxidation of hydrocarbons can generally be represented as:

CₙHₘ+½nO₂ànCO+m/2H₂

For methane, this becomes:

CH₄+½O₂àCO+2H₂ ΔH° 298K=−35.6 KJ/mol

In an exemplary embodiment, one or more waste streams described above are combined into a common waste stream in line 424 and heat exchanged in a pre-heat exchanger 440 to produce a heated common waste stream in line 425. The heated common waste stream in line 425 is then fed to the partial oxidation reactor 420 with a bed 421 of partial oxidation catalyst at the bottom of the reactor. An oxygen stream in line 418 is provided and introduced to the partial oxidation reactor 420 in less than stoichiometric proportions to achieve only partial oxidation of the hydrocarbons and oxygenates to carbon monoxide. The effluent from partial oxidation reactor 420 exits in the partial oxidation reactor effluent stream in line 427 and is sent to the pre-heat exchanger 440 to provide heat to the common waste stream in line 424, then to a fractionator reboiler exchanger 442 to provide heat to a heat exchanger supplying reboiler vapor to the process, and then perhaps to a steam generator 444 to recover any remaining heat from the stream. A partial oxidation syngas stream rich in carbon monoxide is recovered in line 422 at about 177° C. (350° F.) to about 232° C. (450° F.).

The partial oxidation reactor 420 can be a thermal partial oxidation reactor without a catalyst, or it could be a catalytic partial oxidation reactor, with a partial oxidation catalyst bed 421. In a preferred embodiment of the present disclosure, the common waste stream in line 424 is typically free of contaminants such as sulfur so catalytic partial oxidation is preferred. The partial oxidation catalyst bed 421 may comprise a commercially available nickel on alumina catalyst or equivalent. The partial oxidation reactor 420 would operate at temperatures between about 840° F. (450° C.) to about 1470° F. (800° C.).

Boiler feed water in line 430 may be fed to the cold side of a steam generator 444 and steam and water are generated in stream 432.

In an embodiment, a fractionator bottoms stream in line 428 is fed to the fractionator reboiler 442 and partially vaporized therein to produce a heated fractionator bottoms stream in line 426. In an exemplary embodiment, the fractionator bottoms stream in line 428 comprises a portion or all of a second combined cooled circulating jet fractionator bottoms stream in line 413 of FIG. 8 and the heated fractionator bottoms stream in line 426 is returned to the jet fractionator 400.

In a further embodiment of the present disclosure, additional oxygen can be added in the oxygen stream in line 418 in order to oxidize the syngas gas beyond carbon monoxide and hydrogen in order to provide additional heat to the heated fractionator bottoms stream in line 426. In a specific embodiment, the enthalpy provided by fractionator reboiler 442 is all of the duty required by the jet fractionator reboiler heater 416 in FIG. 8. In such a case, the fired heater 416 in FIG. 8 can be eliminated.

The resultant syngas can be recycled into the methanol synthesis unit in FIG. 4. The partial oxidation stream in line 422 may be fed into line 122 of FIG. 4 for use in the synthesis of methanol.

### Reforming

A syngas stream comprising carbon oxides and hydrogen may be produced by reforming the various gaseous waste streams with steam in a steam reforming reactor, an autothermal reforming reactor, or a dry reforming reactor.

### Steam Reforming

Steam reforming the various gaseous waste streams at high temperatures with carbon dioxide and steam will create a syngas mixture that can be fed directly to methanol synthesis. This is an endothermic process so requires a substantial amount of external heat. Steam reforming of hydrocarbons can generally be represented with the chemical equation:

CₙHₘ+nH₂O→nCO+(n+m/2)H₂

For methane, this becomes:

CH₄+H₂O→CO+3H₂ ΔH° 298K=+206 KJ/mol

The various gaseous waste streams are mixed with one to two carbon equivalents of carbon dioxide to prevent elemental carbon production. The gaseous waste stream and carbon dioxide mixture is then mixed with steam to produce a mixture containing about 1 mol carbon from hydrocarbons to 1-2 moles water. The gas/steam mixture enters a steam reforming process unit charged with a commercially available nickel catalyst. The conversion of the hydrocarbons to carbon monoxide and hydrogen takes place at about 700° C. to about 900° C. at a system pressure that may be between 82 kPa (abs) (12 psia) and about 3.5 MPa(abs) (500 psia). A typical flow rate for such a reactor would be about 300 lbs/hr/cubic foot of catalyst. After condensation of the water the product gas from steam reforming may be fed directly to the methanol synthesis section 111 via the syngas stream in line 122 in FIG. 4.

### Autothermal Reforming

Autothermal reforming combines both partial oxidation and steam reforming. Oxygen from the hydrolysis unit, steam, and carbon dioxide are reacted over a catalyst to produce an appropriate syngas mixture for conversion to methanol.

The various gaseous waste streams mentioned above are reacted in an autothermal reactor with a scarcity of oxygen to produce a recycle syngas stream comprising largely carbon monoxide and hydrogen. Heat from the exothermic partial oxidation helps drive the endothermic steam reforming process. The resultant syngas can be recycled into the methanol synthesis unit.

### Dry Reforming

Dry reforming is an endothermic reaction between methane and carbon dioxide to produce carbon monoxide and hydrogen. The various gaseous waste streams mentioned above are reacted in a dry reformer to produce syngas. The resultant syngas can be recycled into the methanol synthesis unit.

The syngas stream of the present disclosure may have a H2/CO ratio between about 0.5 and about 4, preferably about 2.

### Example

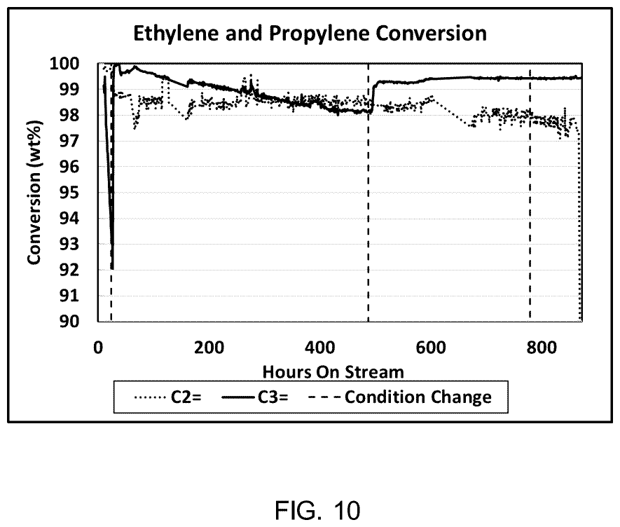

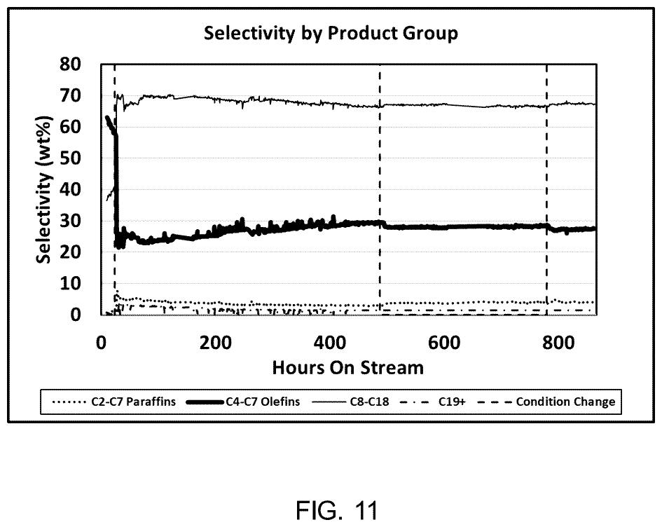

A first stage oligomerization catalyst comprising zeolite and a second stage oligomerization catalyst comprising metal were loaded in a pilot plant reactor. The reactor was fed with a mixed light olefin feed. The oligomerization test was conducted at 6.2 MPa(gauge) (900 psig) pressure, with an inlet temperature to the first oligomerization stage catalyst varying from 160 to 250° C. and to the second oligomerization stage catalyst varying from 130 to 250° C. and WHSV in the fresh olefin charge varying from 0.5 to 1.0 hr⁻¹. Depending on the test conditions, the results demonstrate over 97 wt % ethylene and propylene conversion as shown in FIG. 10 and high selectivity to jet fuel range compounds C8-C18 in FIG. 11. The jet fuel properties were measured and measured properties and their values are listed in the Table below.

TABLE

| Jet Fuel Property | Value |
|---|---|
| Density @ 60 F. | 768.3 kg/m³ |
| Flash Point | 42° C. |
| Freeze Point | <−80° C. |
| Distillation Temp (D86): T10 | 164.3 |
| Distillation Temp (D86): T50 | 197 |
| Distillation Temp (D86): T90 | 263.7 |
| [symbol] lation Temp (D86): Final boiling point | 285.6 |

[symbol]

indicates data missing or illegible when filed

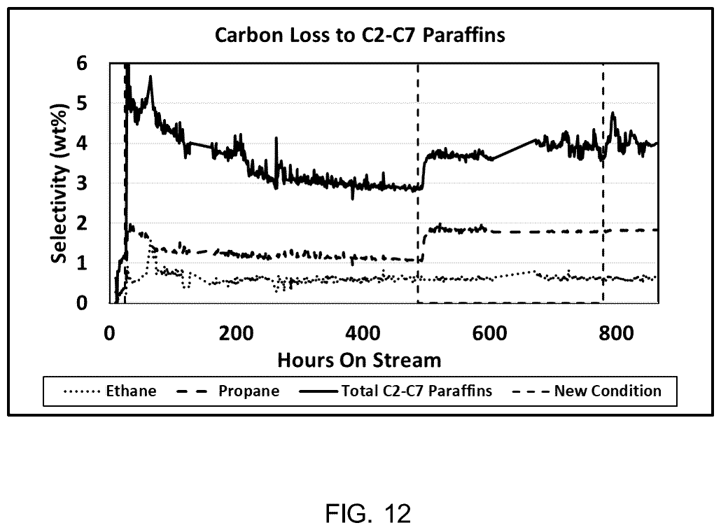

Further, some yield losses to light paraffins were measured on the order of 3-5 wt %, as shown in FIG. 12. The majority of the losses were to ethane and propane. The carefully designed yield structure demonstrated here provides high jet fuel yield and low carbon intensity when integrated within the eFuels complex as described in the present disclosure.

### Specific Embodiments

While the following is described in conjunction with specific embodiments, it will be understood that this description is intended to illustrate and not limit the scope of the preceding description and the appended claims.

A first embodiment of the disclosure is a process for production of a liquid fuel from carbon dioxide and hydrogen, comprising (a) reacting a mixture of carbon dioxide and hydrogen to produce a methanol stream, a water stream and one or more first gaseous waste streams; (b) contacting the methanol with an MTO catalyst to produce one or more olefin streams and a second gaseous waste stream; (c) oligomerizing one or more of the olefin streams with one or more oligomerization catalysts to produce an oligomerized olefin stream and a third gaseous waste stream; (d) hydrogenating the oligomerized olefin stream with a hydrogenation catalyst in the presence of hydrogen to produce a naphtha stream, a jet fuel stream, a diesel stream, and a fourth gaseous waste stream; (e) producing a syngas stream comprising carbon oxides and hydrogen by (1) reforming one or more of the naphtha stream, diesel stream, first gaseous waste stream, second gaseous waste stream, third gaseous waste stream, or fourth gaseous waste stream with steam in a steam reforming reactor, an autothermal reforming reactor, or a dry reforming reactor; or (2) partially oxidizing one or more of the naphtha stream, diesel stream, first gaseous waste stream, second gaseous waste stream, third gaseous waste stream, or fourth gaseous waste stream. An embodiment of the disclosure is one, any or all of prior embodiments in this paragraph up through the first embodiment in this paragraph wherein the syngas stream is recycled to step (a). An embodiment of the disclosure is one, any or all of prior embodiments in this paragraph up through the first embodiment in this paragraph wherein the hydrogen in step (a) is green hydrogen. An embodiment of the disclosure is one, any or all of prior embodiments in this paragraph up through the first embodiment in this paragraph wherein the carbon oxides comprise CO and CO2. An embodiment of the disclosure is one, any or all of prior embodiments in this paragraph up through the first embodiment in this paragraph wherein the syngas stream of step (e) has a H2/CO ratio between about 0.5 and about 4. An embodiment of the disclosure is one, any or all of prior embodiments in this paragraph up through the first embodiment in this paragraph wherein the alkane stream comprises methane, ethane and/or propane. An embodiment of the disclosure is one, any or all of prior embodiments in this paragraph up through the first embodiment in this paragraph wherein the methanol synthesis process, MTO process, and/or oligomerization process produces steam and the steam is used in reforming step (c). An embodiment of the disclosure is one, any or all of prior embodiments in this paragraph up through the first embodiment in this paragraph wherein step (a) takes place the presence of a catalyst. An embodiment of the disclosure is one, any or all of prior embodiments in this paragraph up through the first embodiment in this paragraph wherein the liquid fuel stream of step (d) comprises a naphtha stream, a jet stream, and a diesel fuel stream.

A second embodiment of the disclosure is a process for production of a liquid fuel from carbon dioxide and hydrogen, comprising (a) reacting a mixture of carbon dioxide and hydrogen to produce a crude methanol stream, containing methanol, water, and other contaminants comprising one or more of hydrogen, CO, CO2, methane, ethanol, and other oxygenated hydrocarbons; (b) purifying the crude methanol stream by means of distillation to remove light contaminants into a first gaseous waste stream; heavy contaminants and water into a second heavy waste stream; and to produce a refined methanol stream; (c) contacting the refined methanol stream with an MTO catalyst to produce a crude olefin stream containing ethylene, propylene, butylenes, and other contaminants comprising one or more of hydrogen, CO, CO2, methane, dimethyl ether, ethanol, and other oxygenated hydrocarbons; (d) purifying the crude olefin stream by means of distillation to remove light ends into a third gaseous waste stream; followed by water absorption to remove heavy oxygenated hydrocarbons and water extraction to remove dimethyl ether into a DME (dimethyl ether) recycle stream; and to produce one or more refined olefin streams; (e) reacting the one or more of the refined olefin streams with one or more oligomerization catalysts using one or more reaction vessels to produce a crude oligomerized olefin stream comprising oligomerized olefins with carbon lengths between 4 and 28 carbons and contaminants comprising one or more of hydrogen, methane, and alkanes lighter than pentane; (f) fractionating the crude oligomerized olefin stream by means of distillation to remove hydrogen and light alkanes into (1) a fourth gaseous waste stream, (2) a light oligomerized olefin recycle stream, and (3) a refined oligomerized olefin stream comprising olefins having carbon lengths between 8 and 28 carbons; (g) reacting the refined oligomerized olefin stream with hydrogen with a hydrogenation catalyst to saturate the olefins to paraffins to create a crude jet fuel stream; (h) fractionating the crude jet fuel stream by means of distillation to remove excess hydrogen and light hydrocarbons into (1) a fifth gaseous waste stream, (2) a first liquid waste stream comprising naphtha, (3) a second liquid waste stream comprising diesel, and (4) a liquid fuel comprising jet fuel; (i) producing a recycle syngas stream comprising carbon oxides and hydrogen by (1) reforming one or more of the waste streams with steam in a steam reforming process, an autothermal reforming process, or a dry reforming process; or (2) partially oxidizing one or more of the waste streams; and (j) co-feeding the recycle syngas stream to the methanol synthesis process of step A. An embodiment of the disclosure is one, any or all of prior embodiments in this paragraph up through the second embodiment in this paragraph, further comprising reacting one or more of the waste streams with oxygen in a partial oxidation reactor to produce a second recycle syngas stream comprising CO and hydrogen. An embodiment of the disclosure is one, any or all of prior embodiments in this paragraph up through the second embodiment in this paragraph, wherein the reaction of step (i) is carried out by reacting one or more of the waste streams with oxygen and steam in a an autothermal reactor to produce a third recycle syngas stream comprising CO and hydrogen. An embodiment of the disclosure is one, any or all of prior embodiments in this paragraph up through the second embodiment in this paragraph, wherein the reaction of step (i) is carried out by reacting the first liquid waste stream with steam in a steam reforming process. An embodiment of the disclosure is one, any or all of prior embodiments in this paragraph up through the second embodiment in this paragraph, wherein the reaction of step (i) is carried out by reacting the second liquid waste stream with steam in a steam reforming process. An embodiment of the disclosure is one, any or all of prior embodiments in this paragraph up through the second embodiment in this paragraph, wherein the reaction of step (i) is carried out by partial oxidation and the resulting recycle syngas stream is thermally integrated with the fractionation process of step h) in order to provide some or all of the energy required for the distillation. An embodiment of the disclosure is one, any or all of prior embodiments in this paragraph up through the second embodiment in this paragraph, wherein the hydrogen for steps (a) and/or (g) is produced by a water electrolysis unit. An embodiment of the disclosure is one, any or all of prior embodiments in this paragraph up through the second embodiment in this paragraph, wherein the reaction of step (i) is carried out by partial oxidation and wherein the oxygen for partial oxidation is produced by a water electrolysis unit. An embodiment of the disclosure is one, any or all of prior embodiments in this paragraph up through the second embodiment in this paragraph, wherein the oxygen for autothermal reforming is produced by a water electrolysis unit. An embodiment of the disclosure is one, any or all of prior embodiments in this paragraph up through the second embodiment in this paragraph, wherein the reaction of step (i) is carried out with steam in a steam reforming process and the waste stream or streams fed to the steam reforming reactor contain between about 10 wt % and about 50 wt % propane. An embodiment of the disclosure is one, any or all of prior embodiments in this paragraph up through the second embodiment in this paragraph, wherein the reaction of step (i) is carried out using a dry reforming process.

A third embodiment of the present disclosure is a process for production of a liquid fuel from carbon oxides and hydrogen, comprising (a) reacting a mixture of carbon oxides and hydrogen to produce a methanol stream, a water stream and one or more first gaseous waste streams; (b) contacting the methanol with an MTO catalyst to produce one or more olefin streams and a second gaseous waste stream; (c) oligomerizing one or more of the olefin streams with one or more oligomerization catalysts to produce an oligomerized olefin stream and a third gaseous waste stream; (d) hydrogenating the oligomerized olefin stream with a hydrogenation catalyst in the presence of hydrogen to produce a jet fuel stream and a fourth gaseous waste stream; (e) producing a syngas stream comprising carbon oxides and hydrogen by (1) partially oxidizing one or more of the first gaseous waste streams, the second gaseous waste stream, the third gaseous waste stream, or the fourth gaseous waste stream; or (2) reforming one or more of the first gaseous waste streams, the gaseous second waste stream, the third gaseous waste stream, or the fourth gaseous waste stream with steam in a steam reforming reactor, an autothermal reforming reactor, or a dry reforming reactor. An embodiment of the present disclosure is one, any or all of prior embodiments in this paragraph up through the third embodiment in this paragraph wherein said syngas stream of step (e) has a H₂/CO ratio between about 0.5 and about 4.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for production of a liquid fuel from carbon oxides and hydrogen, comprising:
(a) reacting a mixture of carbon oxides and hydrogen to produce a methanol stream, a water stream and one or more first gaseous waste streams;
(b) contacting said methanol with an MTO catalyst to produce one or more olefin streams and a second gaseous waste stream;
(c) oligomerizing one or more of said olefin streams with one or more oligomerization catalysts to produce an oligomerized olefin stream and a third gaseous waste stream;
(d) hydrogenating said oligomerized olefin stream with a hydrogenation catalyst in the presence of hydrogen to produce a naphtha stream, a jet fuel stream, a diesel stream, and a fourth gaseous waste stream;
(e) producing a syngas stream comprising carbon oxides and hydrogen by
(1) partially oxidizing one or more of said naphtha stream, said diesel stream, said first gaseous waste streams, said second gaseous waste stream, said third gaseous waste stream, or said fourth gaseous waste stream; or
(2) reforming one or more of said naphtha stream, said diesel stream, said first gaseous waste stream, said second gaseous waste stream, said third gaseous waste stream, or said fourth gaseous waste stream with steam in a steam reforming reactor, an autothermal reforming reactor, or a dry reforming reactor.

2. The process of claim 1 wherein said syngas stream is recycled to step (a).

3. The process of claim 1 wherein said hydrogen in step (a) is green hydrogen.

4. The process of claim 1 wherein said carbon oxides comprise CO and CO₂.

5. The process of claim 4 wherein said syngas stream of step (e) has a H₂/CO ratio between about 0.5 and about 4.

6. The process of claim 1 wherein said methanol synthesis process, MTO process, and/or oligomerization process produces steam and said steam is used in reforming step (e).

7. The process of claim 1 wherein step (a) takes place in the presence of a catalyst.

8. A process for production of a liquid fuel from carbon oxides and hydrogen, comprising:
(a) reacting a mixture of carbon oxides and hydrogen to produce a crude methanol stream, containing methanol, water, and other contaminants comprising one or more of hydrogen, CO, CO₂, methane, ethanol, and other oxygenated hydrocarbons;
(b) purifying said crude methanol stream by means of distillation to remove light contaminants into a first gaseous waste stream; heavy contaminants and water into a second heavy waste stream; and to produce a refined methanol stream;
(c) contacting said refined methanol stream with an MTO catalyst to produce a crude olefin stream containing ethylene, propylene, and other contaminants comprising one or more of hydrogen, CO, CO₂, methane, dimethyl ether, ethanol, and other oxygenated hydrocarbons;
(d) purifying said crude olefin stream by means of distillation to remove light ends into a third gaseous waste stream; followed by water absorption to remove heavy oxygenated hydrocarbons and water extraction to remove dimethyl ether; and to produce one or more refined olefin streams;
(e) reacting one or more of said refined olefin streams with one or more oligomerization catalysts using one or more reaction vessels to produce a crude oligomerized olefin stream comprising oligomerized olefins with carbon lengths between 4 and 28 carbons and contaminants comprising one or more of hydrogen, methane, and alkanes lighter than pentane;
(f) fractionating said crude oligomerized olefin stream by means of distillation to remove hydrogen and light alkanes into (1) a fourth gaseous waste stream, (2) a light oligomerized olefin recycle stream, and (3) a refined oligomerized olefin stream comprising olefins having carbon lengths between 8 and 28 carbons;
(g) reacting said refined oligomerized olefin stream with hydrogen with a hydrogenation catalyst to saturate the olefins to paraffins to create a crude jet fuel stream;
(h) fractionating said crude jet fuel stream by means of distillation to remove excess hydrogen and light hydrocarbons into (1) a fifth gaseous waste stream, (2) a first liquid waste stream comprising naphtha, (3) a second liquid waste stream comprising diesel, and (4) a liquid fuel comprising jet fuel;
(i) producing a recycle syngas stream comprising carbon oxides and hydrogen by
(1) partially oxidizing one or more of said waste streams; or
(2) reforming one or more of said waste streams with steam in a steam reforming process, an autothermal reforming process, or a dry reforming process; and
(j) co-feeding said recycle syngas stream to the methanol synthesis process of step (a).

9. The process of claim 8, further comprising reacting one or more of the waste streams with oxygen in a partial oxidation reactor to produce a second recycle syngas stream comprising carbon oxides and hydrogen.

10. The process of claim 8, wherein the reaction of step (i) is carried out by reacting one or more of the waste streams with oxygen and steam in an autothermal reactor to produce a third recycle syngas stream comprising carbon oxides and hydrogen.

11. The process of claim 8, wherein the reaction of step (i) is carried out by reacting the first liquid waste stream with steam in a steam reforming process.

12. The process of claim 8, wherein the reaction of step (i) is carried out by reacting the second liquid waste stream with steam in a steam reforming process.

13. The process of claim 8, wherein the reaction of step (i) is carried out by partial oxidation and the resulting recycle syngas stream is thermally integrated with the fractionation process of step (h) in order to provide some or all of the energy required for the distillation.

14. The process of claim 8, wherein the hydrogen for steps (a) and/or (g) is produced by a water electrolysis unit.

15. The process of claim 8, wherein the reaction of step (i) is carried out by partial oxidation and wherein the oxygen for partial oxidation is produced by a water electrolysis unit.

16. The process of claim 8, wherein the oxygen for autothermal reforming is produced by a water electrolysis unit.

17. The process of claim 8, wherein the reaction of step (i) is carried out with steam in a steam reforming process and the waste stream or streams fed to the steam reforming reactor contain between about 10 wt % and about 50 wt % propane.

18. The process of claim 8, wherein the reaction of step (i) is carried out using a dry reforming process.

19. A process for production of a liquid fuel from carbon oxides and hydrogen, comprising:
(a) reacting a mixture of carbon oxides and hydrogen to produce a methanol stream, a water stream and one or more first gaseous waste streams;
(b) contacting said methanol with an MTO catalyst to produce one or more olefin streams and a second gaseous waste stream;
(c) oligomerizing one or more of said olefin streams with one or more oligomerization catalysts to produce an oligomerized olefin stream and a third gaseous waste stream;
(d) hydrogenating said oligomerized olefin stream with a hydrogenation catalyst in the presence of hydrogen to produce a jet fuel stream and a fourth gaseous waste stream;
(e) producing a syngas stream comprising carbon oxides and hydrogen by
(1) partially oxidizing one or more of said first gaseous waste streams, said second gaseous waste stream, said third gaseous waste stream, or said fourth gaseous waste stream; or
(2) reforming one or more of said first gaseous waste streams, said gaseous second waste stream, said third gaseous waste stream, or said fourth gaseous waste stream with steam in a steam reforming reactor, an autothermal reforming reactor, or a dry reforming reactor.

20. The process of claim 19 wherein said syngas stream of step (e) has a H2/CO ratio between about 0.5 and about 4.
